(19) United States
(12) Patent Application Publication
(10) Pub. No.: US 2023/0150922 A1
(43) Pub. Date: May 18, 2023

(54) Fluorinated Derivatives of Gabapentin and Methods of Use Thereof

(71) Applicant: The General Hospital Corporation, Boston, MA (US)

(72) Inventors: Pedro Brugarolas, Ithaca, NY (US); YuPeng Zhou, East Boston, MA (US)

(21) Appl. No.: 17/919,848

(22) PCT Filed: Apr. 21, 2021
(86) PCT No.: PCT/US2021/028455

§ 371 Date: Oct. 19, 2022

Related U.S. Application Data
(60) Provisional application No. 63/013,430, filed on Apr. 21, 2020.

(51) Int. Cl. C07C 229/32; C07B 39/00; C07C 235/14; C07D 209/54; C07B 59/00; A61K 51/04

(52) U.S. Cl. CPC C07C 229/32; C07B 39/00; C07C 235/14; C07D 209/54; C07B 59/001; C07B 59/002; A61K 51/0402; A61K 51/0446; C07B 2200/05

(57) ABSTRACT

Described herein are fluorinated derivatives of gabapentin and methods of synthesis and methods of use thereof, e.g., in imaging and therapy.

### CLAIM OF PRIORITY

This application claims the benefit of U.S. Provisional Application Ser. No. 63/013,430, filed on Apr. 21, 2020. The entire contents of the foregoing are incorporated herein by reference.

### STATEMENT REGARDING FEDERAL FUNDING

This invention was made with government support under Grant No. NS120139 awarded by the National Institutes of Health. The government has certain rights in the invention.

### TECHNICAL FIELD

Described herein are fluorinated derivatives of gabapentin and methods of synthesis and methods of use thereof.

### BACKGROUND

Neuropathic pain affects 7-10% of the world's population. In addition to the debilitating effects that neuropathic pain causes, chronic pain has contributed to the current opioid epidemic.

### SUMMARY

Described herein are fluorine-containing gabapentin derivatives, which can be radiolabeled for use as PET tracers, e.g., for use in imaging neuropathic pain and other conditions, e.g., neurological conditions such as epilepsy, anxiety and ataxia, as well as for treatment of those conditions.

Provided herein are compounds of Formula (I) or Formula (II):

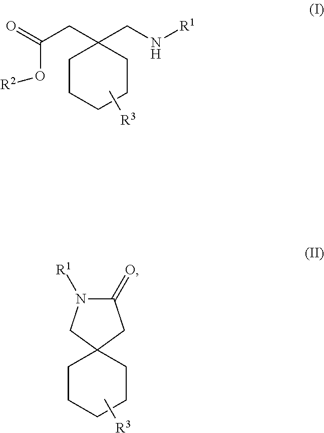

or a pharmaceutically acceptable salt thereof, wherein:
each R¹ is independently selected from H and C(═O)(O—C₁₋₆ alkyl), wherein said C₁₋₆ alkyl is optionally substituted with OC(═O)C₁₋₆ alkyl;

R² is selected from H and C₁₋₆ alkyl, benzyl; and

each R³ is independently a fluorine atom selected from F and ¹⁸F. The compound of claim 1, wherein at least one hydrogen atom is a deuterium isotope.

In some embodiments, at least one carbon atom is an ¹¹C isotope

In some embodiments, the fluorine atom is ¹⁸F.

In some embodiments, the compound of Formula (I) is selected from any one of the following formulae:

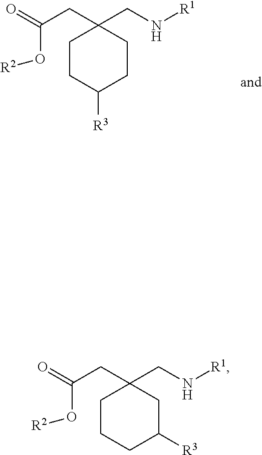

or a pharmaceutically acceptable salt thereof.

In some embodiments, the compound of Formula (II) has any one of the following formulae:

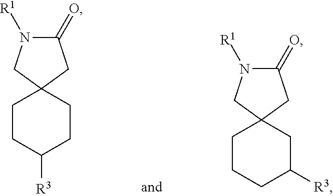

or a pharmaceutically acceptable salt thereof.

In some embodiments, the compound is cis-[¹⁸F]4-fluoro-gabapentin. In some embodiments, the compound is trans-[¹⁸F]4-fluoro-gabapentin.

Also provided herein are pharmaceutical compositions comprising the compounds described herein and a pharmaceutically acceptable carrier. In some embodiments, the compound is cis-[¹⁸F]4-fluoro-gabapentin. In some embodiments, the compound is trans-[¹⁸F]4-fluoro-gabapentin. Further provided are compositions comprising a mixture of cis-[¹⁸F]4-fluoro-gabapentin and trans-[¹⁸F]4-fluoro-gabapentin.

Additionally, provided herein are methods for treating neuropathic pain, epilepsy, anxiety, or ataxia in a subject. The methods include administering a therapeutically effective amount of a compound or composition as described herein to a subject in need thereof. In some embodiments, the compound is cis-[¹⁸F]4-fluoro-gabapentin. In some embodiments, the compound is trans-[¹⁸F]4-fluoro-gabapentin.

In some embodiments, the composition is administered at doses ranging from 0.001 mg/kg to 100 mg/kg.

In some embodiments, the composition is administered orally or intravenously.

Also provided herein are methods for imaging a subject. The methods include administering to a subject a compound (or composition comprising a compound) as described herein, wherein the compound comprises a radioisotope, and detecting the compound in the subject. In some embodiments, the fluorine atom is ¹⁸F. In some embodiments, the compound is cis-[¹⁸F]4-fluoro-gabapentin. In some embodiments, the compound is trans-[¹⁸F]4-fluoro-gabapentin.

In some embodiments, detecting the compound comprises using Positron Emission Tomography (PET) to obtain an image of the compound in the subject.

In some embodiments, the methods further include comparing the image to a reference image, and identifying a subject who has an increased amount of the compound as compared to the reference image, and optionally administering a treatment to the subject. In some embodiments, the treatment comprising administration of gabapentin or a compound or composition as described herein (e.g., a non-radioactive compound).

Further, provided herein are methods for diagnosing neuropathic pain, epilepsy, anxiety, or ataxia in a subject. The methods include administering a detectable amount of a compound as described herein, wherein the compound comprises a radioisotope, to a subject and detecting the compound comprised in the imaging agent in the subject by Positron Emission Tomography (PET). In some embodiments, the fluorine atom is ¹⁸F. In some embodiments, the compound is cis-[¹⁸F]4-fluoro-gabapentin. In some embodiments, the compound is trans-[¹⁸F]4-fluoro-gabapentin.

Also provided are the compounds described herein for use in a method of treating neuropathic pain, epilepsy, anxiety, or ataxia in a subject, the method comprising administering a therapeutically effective amount of the compound to a subject in need thereof. In some embodiments, the compound is cis-[¹⁸F]4-fluoro-gabapentin. In some embodiments, the compound is trans-[¹⁸F]4-fluoro-gabapentin. In some embodiments, the composition is administered at doses ranging from 0.001 mg/kg to 100 mg/kg. In some embodiments, the composition is administered orally or intravenously.

Additionally provided are the compounds described herein for use in a method of imaging a subject, the method comprising administering the compound to a subject and detecting the compound in the subject. In some embodiments, the fluorine atom is ¹⁸F. In some embodiments, the compound is cis-[¹⁸F]4-fluoro-gabapentin. In some embodiments, the compound is trans-[¹⁸F]4-fluoro-gabapentin. In some embodiments, detecting the compound comprises using Positron Emission Tomography (PET) to obtain an image of the compound in the subject. In some embodiments, the image is compared to a reference image, and identifying a subject who has an increased amount of the compound as compared to the reference image, and optionally administering a treatment to the subject. In some embodiments, the treatment comprising administration of gabapentin or a compound as described herein (e.g., a non-radioactive compound).

Further provided are the compounds described herein for use in a method for diagnosing neuropathic pain, epilepsy, anxiety, or ataxia in a subject, the method comprising administering to a subject a compound as described herein, wherein the compound comprises a radioisotope, and detecting the compound comprised in the imaging agent in the subject by Positron Emission Tomography (PET). In some embodiments, the fluorine atom is ¹⁸F. In some embodiments, the compound is cis-[¹⁸F]4-fluoro-gabapentin. In some embodiments, the compound is trans-[¹⁸F]4-fluoro-gabapentin.

Also provided herein are methods of making the compounds of Formula (I):

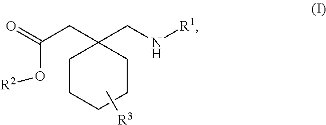

or a pharmaceutically acceptable salt thereof, comprising converting a compound of Formula (II):

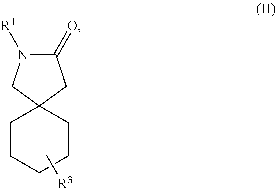

or a pharmaceutically acceptable salt thereof, to afford a compound of Formula (I), or a pharmaceutically acceptable salt thereof.

In some embodiments, the compound of Formula (II), or a pharmaceutically acceptable salt thereof, is prepared by a process comprising reacting a compound of Formula:

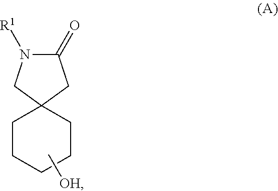

with a fluoride source to afford a compound of Formula (II).

In some embodiments, the compound of Formula (A) is prepared by a process comprising reducing a compound of Formula (B):

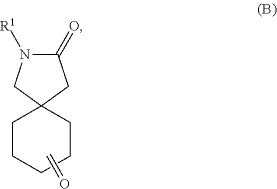

to afford the compound of Formula (A).

The term “radioactive isotope” or “radionuclide” refers to an isotope having an unstable nucleus that decomposes spontaneously by emission of a nuclear electron, positron, or helium nucleus and radiation, thus achieving a more stable nuclear composition.

The term “deuterated version” as used herein means one or more of hydrogen in a compound is replaced with ²H, an isotope of hydrogen.

As used herein, the term “radioactive tracer”, or “radioactive label”, or “tracer”, “radiotracer,” “radioligand,” “PET ligand” or “radiopharmaceutical” means a chemical compound in which one or more atoms have been replaced by a radioisotope. By virtue of its radioactivity, it can be used to explore the mechanism of chemical reactions by tracing the path that the radioisotope follows from reactants to products. A radioactive tracer can also be used to track the distribution of a substance within a natural system such as a cell or tissue. Radioactive tracers, radioligands, and PET ligands form the basis of a variety of imaging systems, such as PET scans and SPECT scans. A radiopharmaceutical includes both radioactive molecules used for imaging and for therapy, e.g., radiotherapy.

The use of the word “a” or “an,” when used in conjunction with the term “comprising” in the claims and/or the specification may mean “one,” but it is also consistent with the meaning of “one or more,” “at least one,” and “one or more than one.”

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

An “isomer” of a first compound is a separate compound in which each molecule contains the same constituent atoms as the first compound, but where the configuration of those atoms in three dimensions differs. In the present compounds, a Cis isomer has CH₂NH₂ and F on the same side, while in the trans isomer they are on opposite sides. See, e.g., Bryans et al., J. Med. Chem. 1998, 41, 1838-1845.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Prodrug” means a compound that is convertible in vivo metabolically into a drug or a tracer according to the present invention. The prodrug itself may or may not also have activity with respect to a given target protein. For example, a compound comprising a hydroxy or carboxylic acid group may be administered as an ester that is converted by hydrolysis in vivo to the hydroxy or carboxylic acid compound. Suitable esters that may be converted in vivo into hydroxy compounds include acetates, citrates, lactates, phosphates, tartrates, malonates, oxalates, salicylates, propionates, succinates, fumarates, maleates, methylene-bis-.beta.-hydroxynaphthoate, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, a compound comprising an amine group may be administered as an amide that is converted by hydrolysis in vivo to the amine compound.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 shows the structures of gabapentin (top) and 4-fluorogabapentin derivatives thereof.

FIGS. 2A-B are exemplary synthetic schemes for 4F-GBP cold (2A) and radioactive (2B).

FIG. 2C is a radiochromatogram of labeling reaction crude containing showing semiprep radioHPLC purification of trans (iso 2) and cis (iso 1) [18F]4F-GBP FIGS. 2D-E show exemplary alternative synthetic schemes for trans-GBP4F and trans-[¹⁸F]4F-GBP (2D) and cis-GBP4F (isomer 1) and cis-[¹⁸F]4FGBP (2E).

FIG. 2F is a pair of radiochromatograms of pure trans-(top) and cis-(bottom) 4F-GBP.

FIG. 2G shows results of coinjection of collected fractions with reference standard (radiodetector and ELS detector).

FIGS. 3A-F show Evaluation of [¹⁸F]4F-GBP. A. Comparison of published α2δ-1 immunostaining (Taylor and Garrido, 2008)⁴⁵ with autoradiography of [³H]gabapentin and [¹⁸F]F-gabapentin in rat brain slices. Areas of high α2δ-1 expression by IHC (shown in reddish-brown) correlate areas of high [³H]/[¹⁸F]-gabapentin binding (darker autoradiographic signal). B. Over 70% of [¹⁸F]F-gabapentin binding can be displaced by non-radiolabeled gabapentin, pregabalin or F-gabapentin. C. [³H]gabapentin binding to axial and longitudinal spinal cord sections from Sham and SNI mice. Compared to Sham, autoradiographic signal is increased 245% in whole longitudinal sections. D. Left: α2δ-1 immunostaining of spinal cord sections⁴⁵. [³H]gabapentin localized to the dorsal horn, consistent with IHC. Center: Zoom-in of axial spinal cord sections. E. Spinal cord autoradiography comparing [¹⁸F]4-fluoro-gabapentin cis and trans isomers. Scale bars 5 mm. F. PET imaging of mice after administration of [¹⁸F]4-fluoro-gabapentin cis and trans isomers

FIGS. 4A-B are graphs showing results of gamma counting in animals that received cis-(5A; control: 1.18±0.13 (n=3) v. disease 1.87±0.41 (n=4)) and trans-[¹⁸F]4F-GBP (5B; control: 1.23±0.15 (n=2) v. disease 2.57±0.34 (n=2)).

FIG. 4C shows autoradiography of L4-L6 explanted spinal nerves from the neuropathic rat model after in vivo administration of [18F]4F-GBP showed that there was significantly higher binding of both cis-[¹⁸F]4F-GBP (top) and trans-[¹⁸F]4F-GBP (bottom) in the injured right nerve (right side) than the left nerve (left side).

FIG. 4D shows that trans-[¹⁸F]4F-GBP had higher binding affinity than gabapentin whereas cis-[¹⁸F]4F-GBP had a lower binding affinity.

FIGS. 5A-B show PET images of [18F]GBP-4F in rhesus macaques, showing significant brain penetration and partial displacement for the cis isomer in the presence of GBP.

FIG. 6A contains an exemplary synthetic scheme for the preparation of prodrug compounds 9a (cold) and 10a (hot) from the parent gabapentin compounds 6a and 8a, respectively.

FIG. 6B contains a schematic illustration for prodrug compound 10a crossing a blood-brain barrier and enzymatic degradation to produce the active gabapentin compound 8a.

FIG. 7 contains a schematic illustration of chemical synthesis of 4F-gabapentin lactam.

FIG. 8 contains a schematic illustration of chemical synthesis of 4F-gabapentin ethyl ester.

FIG. 9 contains a schematic illustration of chemical synthesis of 3/5F-gabapentin.

### DETAILED DESCRIPTION

Developing new nonopioid pain medications is now a major area of interest. An important challenge for developing new pain medications is how to objectively measure that they are working. Positron emission tomography (PET) has the potential to provide specific and quantitative biochemical information about whether a drug is engaging their target in vivo and whether a disease relevant target has increased or decreased as a result of a treatment. However, no PET tracers are known for use in imaging neuropathic pain. One challenge in developing such a PET tracer (endpoint) was to identify a biological target (biomarker) that provides useful physiological information. As shown herein, the expression of the α2δ-1 subunit of voltage gated calcium channels is a robust biomarker for neuropathic pain. This protein is involved in the transmission of pain signals and its expression is greatly increased (3-17 fold) in animal models of pain¹⁻⁴.

To measure α2δ-1, we developed a radiolabeled form of gabapentin, a selective ligand of α2δ-1. This tracer has good properties for PET imaging because gabapentin enters the central nervous system, has high selectivity and affinity for α2δ receptors, and is metabolically stable⁵. Carbon-11 and fluorine-18 are the most common PET isotopes for labeling small molecules. The longer half-life of fluorine-18 (109 min) compared to carbon-11 (20 min) makes it a better candidate for widespread use. Nevertheless, since most drugs do not contain fluorine, developing a ¹⁸F-labeled tracer typically requires lengthy structure-activity relation (SAR) studies in order to identify fluorinated derivatives that retain binding and have adequate pharmacokinetics.

Thus, provided herein are compositions and methods for imaging neuropathic pain using radiolabeled gabapentin. As shown herein, ¹⁸F-labeled 4F-gabapentin binds to α2δ-1 receptors and that binding was increased in the spinal cord of a rat neuropathic pain model by autoradiography and gamma counting. We also showed that 18F-gabapentin enters the brain of mice and monkeys using PET imaging.

These compositions and methods can also be used to measure changes in α2δ-1 expression associated with other disease states such as epilepsy, anxiety, hypertension, cancer, and ataxia⁶, and for treatment of such diseases.

Fluorine-Containing Gabapentin Derivatives

Described herein are fluorogabapentin (F-GBP) and radiofluorinated versions thereof (e.g., [¹⁸F]fluorogabapentin), and derivatives thereof.

In some embodiments, the present disclosure provides a compound of Formula (I):

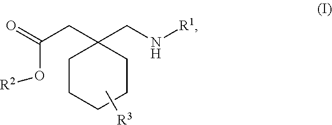

or a pharmaceutically acceptable salt thereof, wherein:
R¹ is selected from H and C(═O)(O—C₁₋₆ alkyl), wherein said C₁₋₆ alkyl is optionally substituted with OC(═O)C₁₋₆ alkyl;
R² is selected from H and C₁₋₆ alkyl and benzyl; and
R³ is a fluorine atom selected from F and ¹⁸F.

In some embodiments, the compound of Formula (I) has formula:

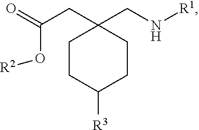

or a pharmaceutically acceptable salt thereof.

In some embodiments, the compound of Formula (I) has formula:

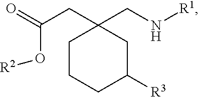

or a pharmaceutically acceptable salt thereof.

In some embodiments, the compound of Formula (I) has formula:

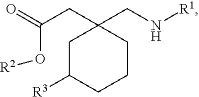

or a pharmaceutically acceptable salt thereof.

In some embodiments, the compound of Formula (I) is selected from any one of the following compounds:

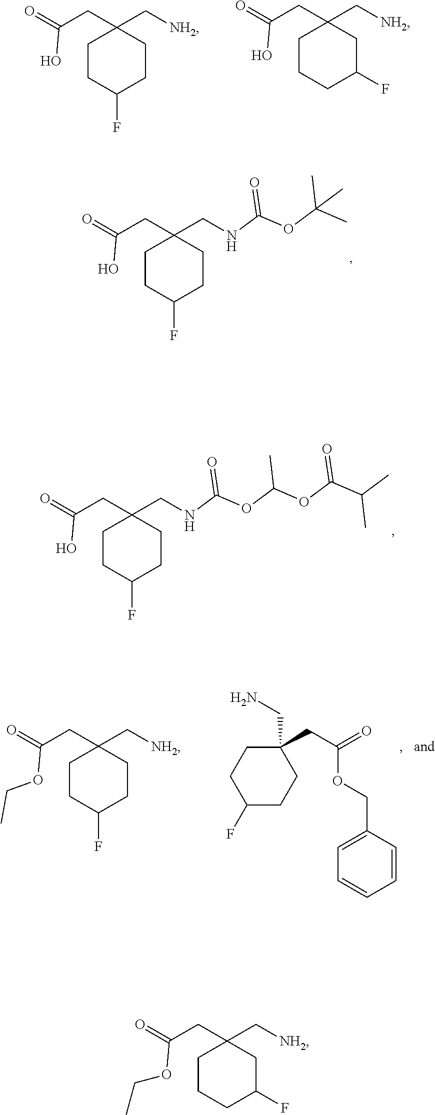

or a pharmaceutically acceptable salt thereof.

In some embodiments, the compound of Formula (I) is selected from any one of the following compounds:

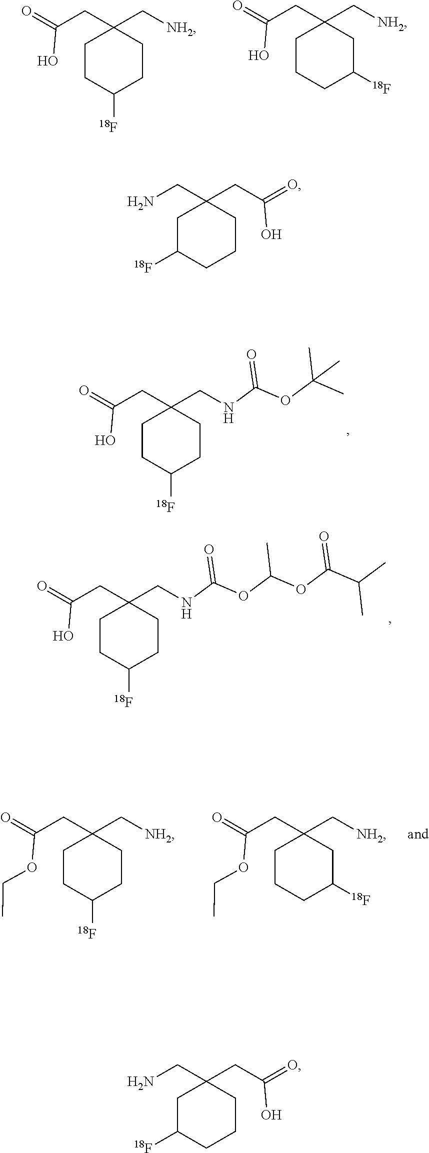

or a pharmaceutically acceptable salt thereof.

In some embodiments, the present disclosure provides a compound of Formula (II):

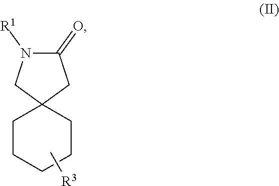

or a pharmaceutically acceptable salt thereof, wherein:
R¹ is selected from H and C(═O)(O—C₁₋₆ alkyl), wherein said C₁₋₆ alkyl is optionally substituted with OC(═O)C₁₋₆ alkyl; and
R³ is a fluorine atom selected from F and ¹⁸F.

In some embodiments, the compound of Formula (II) has formula:

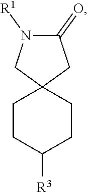

or a pharmaceutically acceptable salt thereof.

In some embodiments, the compound of Formula (II) has formula:

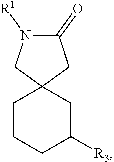

or a pharmaceutically acceptable salt thereof.

In some embodiments, the compound of Formula (II) has formula:

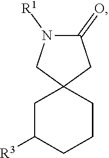

or a pharmaceutically acceptable salt thereof.

In some embodiments, the compound of Formula (II) is selected from any one of the following compounds:

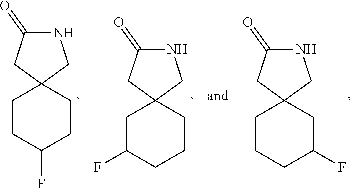

or a pharmaceutically acceptable salt thereof.

In some embodiments, the compound of Formula (II) is selected from any one of the following compounds:

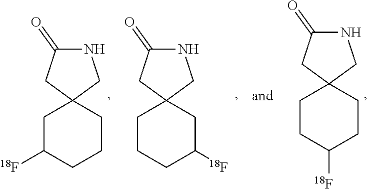

or a pharmaceutically acceptable salt thereof.

Described herein are 4-fluorogabapentin (4F-GBP) and radiofluorinated versions thereof (e.g., 4-[¹⁸F]fluorogabapentin). In some embodiments, the 4-fluorogabapentin is a mixture of cis/trans 4-fluorogabapentin; alternatively the 4-fluorogabapentin is isomerically pure, e.g., at least 80%, 85%, 90% 95%, 97%, 99%, or 100% pure cis or trans 4-fluorogabapentin. These compounds can be made using the methods outlined in the summary of the invention section and in the Examples section. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

Methods of Making Gabapentin Derivatives

The compounds or compositions described herein can also be prepared by any of the applicable techniques of organic synthesis and polymer chemistry. Many such techniques are well known in the art. Many of the known techniques are elaborated in Compendium of Organic Synthetic Methods, Vol 1, 1971; Vol. 2, 1974; Vol. 3, 1977; Vol. 4, 1980; Vol. 5, 1984; and Vol. 6, 1985; Comprehensive Organic Synthesis Selectivity, Strategy & Efficiency in Modern Organic Chemistry, 1993; Advanced Organic Chemistry, Part B: Reactions and Synthesis, 4.sup.th Ed., 2001; Advanced Organic Chemistry, Reactions, Mechanisms, and Structure, 2.sup.nd Ed., 1977; Protecting Groups in Organic Synthesis, 2.sup.nd Ed., 991; Comprehensive Organic Transformations, 2.sup.nd Ed., 1999, Textbook of Polymer Chemistry, 3.sup.rd Ed., 1984, Organic Polymer Chemistry, 2.sup.nd Ed., 1973, and Polymer Science, 1986. These are incorporated herein by reference.

In some embodiments, the present disclosure provides a method of making the compound of Formula (I):

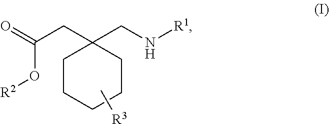

or a pharmaceutically acceptable salt thereof, comprising converting a compound of Formula (II):

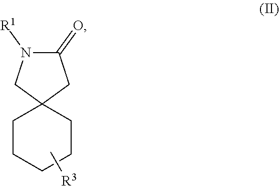

or a pharmaceutically acceptable salt thereof, to afford a compound of Formula (I), or a pharmaceutically acceptable salt thereof.

In some embodiments, the compound of Formula (II), or a pharmaceutically acceptable salt thereof, is prepared by a process comprising reacting a compound of Formula:

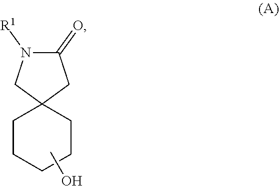

with a fluoride source to afford a compound of Formula (II).

In some embodiments, the compound of Formula (A) is prepared by a process comprising reducing a compound of Formula (B):

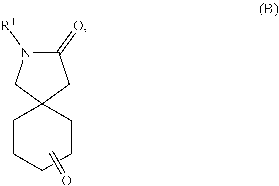

to afford the compound of Formula (A).

Also provided herein are pharmaceutical compositions comprising the 4-fluorogabapentin and radiofluorinated versions thereof, and methods of making the same.

Methods of Diagnosis

The expression of the α2δ-1 subunit of voltage-gated calcium channels is a robust biomarker of neuropathic pain. This protein is involved in nociception and its expression is highly elevated (3-17 fold) in the spinal cord and ganglia in many models of neuropathic pain^{1-4,7-13}.

α2δ is one of the auxiliary subunits of voltage-dependent calcium channels¹⁹. The expression of α2δ-1 is highly increased in experimental models of neuropathic pain where it correlates with the development of allodynia and hyperalgesia. For example: Luo et al described a >17-fold increase in α2δ-1 in dorsal root ganglia (DRGs) from rats with spinal nerve ligation¹; Luo also showed a 10-fold increase in a model of spinal nerve transection and a 5-fold increase in a model of sciatic nerve chronic constriction injury⁷; Newtown et al described a 3-fold increase in the affected DRG in rats with unilateral partial sciatic nerve ligation compared to the contralateral side²; Bauer et al described a 5-fold increase in the superficial and deeper layers of the spinal cord dorsal horn in rats that had undergone spinal nerve ligation³; additional studies have shown 2-5 fold increases in models of trigeminal neuralgia^{8,9}, spinal cord contusion injury^{10,11}, vincristine-induced peripheral neuropathy¹², and paclitaxel-induced neuropathic pain¹³. In all these models, the increases in α2δ-1 expression occur several days following injury and correlate with the development of hyperalgesia or allodynia. In addition, transgenic mice overexpressing α2δ-1 display a phenotype of hyperalgesia²⁰. Given the fold change and the strong association between neuropathic pain and α2δ-1 expression, described herein are methods of imaging α2δ-1 upregulation with PET using a radiofluorinated form of gabapentin, a selective ligand of α2δ-1. As shown herein, this radioligand has good properties for PET imaging because it enters the central nervous system, it is metabolically stable and has high selectivity and affinity for α2δ receptors. PET radioligands have been developed for imaging potassium channels in the brain based on an existing drug¹⁴⁻¹⁸.

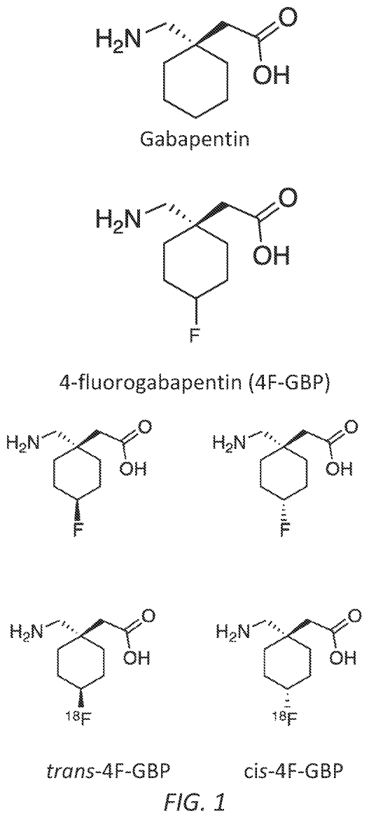

Gabapentin (FIG. 1) is structurally related to Gamma aminobutyric acid (GABA), a naturally occurring amino acid that works as a neurotransmitter in the CNS. Gabapentin, though originally developed as a brain permeable GABA agonist, however, does not bind to GABA_{A} or GABA_{B} receptors but instead binds to α2δ receptors²¹. Evidence for selective binding of pregabalin and gabapentin to α2δ includes isolation of α2δ-1 as the gabapentin binding protein from rat brain membranes²², in vitro binding studies^{23,24}, autoradiography results²⁵, a receptor panel screen²⁶ as well as in vivo evidence demonstrating that a mouse with a point mutation in α2δ-1 protein (R217A) is insensitive to pregabalin^{27,28}.

Treatment with gabapentinoids of animal models of pain results in decreased sensitivity to pain. In addition, sustained treatment with gabapentinoids in animal models reduces the cell-surface expression of α2δ-1 concurrently with the decrease in pain sensitivity 2. In humans, gabapentin and pregabalin are effective therapeutics for neuropathic pain^{30,31}. In clinical trials, oral gabapentin was associated with decreases in self-reported pain in 46% of patients with postherpetic neuralgia and 52% of patients with diabetic neuropathy³². A study in patients with trigeminal neuralgia reported a reduction in facial pain in 43 out of 92 subjects (46%)³³. Several mechanisms have been proposed to explain how gabapentinoids reduce pain. These mechanisms include inhibiting voltage-dependent calcium channels^{34,35}, inhibiting the interaction between α2δ-1 and thrombospondins^{36,37} and inhibiting the interaction between α2δ-1 and NMDA receptors¹³. It is currently unknown why gabapentin does not work to treat pain in every patient, but it is likely that other pathways may be involved. Therefore, the PET tracers described herein that are based on gabapentin can be used to identify those that are likely to respond to gabapentin and provide a method for measuring target engagement of gabapentinoid drugs in clinical studies and changes in α2δ levels due to treatment.

The fluorine-containing gabapentin derivatives described herein can be used as diagnostic imaging agents, e.g., for positron emission tomography (PET). PET is a non-invasive medical imaging technique that relies on the detection of radiation emitted by a radionuclide (radioactive tracer) introduced in the body of the subject on a biologically active molecule. In some embodiments, the compounds are administered at 0.1 to 30 mCi, and are administered intravenously. Images of the radioactive tracer's localization can be reconstructed by computer analysis providing quantitative maps of the radioactive tracer's distribution in the body of the subject. Such images can provide valuable information of the biochemistry and physiology of a subject. Because PET is a molecular imaging technique, it can detect cellular abnormalities. See, e.g., US 20210017133.

Other medical imaging techniques include, but are not limited to, radiography, magnetic resonance imaging (MRI), fiduciary markers, nuclear medicine, photo acoustic imaging, tomography, and ultrasound. See, e.g., US 20210017133.

The compounds described herein can thus be used as imaging agents in medical imaging applications. For example, the compounds can be used to image pathological changes (i.e., increased expression of α2δ receptors) associated with neuropathic pain. Currently, there is no test that can locate and measure pain with precision. The use of these compounds may serve to better diagnose neuropathic pain and to monitor the response to treatment. In addition, these compounds are useful for measuring the capacity of new drugs that act at α2δ receptors to bind to their target, in humans and animals. Finally, these compounds can be used to monitor the ability of new drugs for neuropathic pain to alter the expression of α2δ receptors in clinical trials, which is indicative of pain. Other potential uses of these compounds include the diagnosis and monitoring of other neurological diseases such as epilepsy, anxiety, and ataxia, since gabapentin and pregabalin are effective therapeutics for these conditions. Importantly, these compounds provide a method to visualize underlying pathological changes in these diseases without the need of an acute episode.

In some embodiments, the methods include determining a level of expression of α2δ receptors in a subject, e.g., in a tissue of the subject, and comparing the presence and/or level with one or more references, e.g., a control reference that represents a normal level of expression of α2δ receptors, e.g., a level in an unaffected subject or in an unaffected tissue of the same subject, and/or a disease reference that represents a level of expression of α2δ receptors associated with neuropathic pain, anxiety, epilepsy, or ataxia, e.g., a level in a subject having neuropathic pain, anxiety, epilepsy, or ataxia. If the level of expression of α2δ receptors is comparable to the presence and/or level of the protein(s) in the disease reference, and the subject has one or more symptoms associated with the disease, then the subject can be diagnosed with the disease.

The present methods can also be used to choose a treatment for a subject who has neuropathic pain, anxiety, epilepsy, or ataxia; once it has been determined that a person who has neuropathic pain, anxiety, epilepsy, or ataxia, and also has levels of expression of α2δ receptors above a reference, then a treatment, e.g., comprising gabapentin or pregabalin, as known in the art, or a 4F-GBP as described herein, can be administered.

Suitable reference values can be determined using methods known in the art, e.g., using standard clinical trial methodology and statistical analysis. The reference values can have any relevant form.

The predetermined level can be a single cut-off (threshold) value, such as a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

In some embodiments, the predetermined level is a level or occurrence in the same subject, e.g., at a different time point, e.g., an earlier time point.

Methods of Treatment

The fluorine-containing gabapentin derivatives described herein can be used to treat a number of conditions, including neuropathic pain, epilepsy, anxiety and ataxia, as well as restless legs syndrome (RLS). Neuropathic pain includes but is not limited to painful diabetic neuropathy, HIV-associated neuropathy, chemotherapy-induced neuropathic, postherpetic neuralgia, and trigeminal neuralgia.

Generally, the methods include administering a therapeutically effective amount of a 4F-GBP as described herein, to a subject who is in need of, or who has been determined to be in need of, such treatment. In some embodiments, the methods include the use of a mixture of the cis- and trans-4F-GPB, or a composition that is substantially pure (e.g., at least 80%, 85%, 90%, 95%, or 99%) or 100% pure for one isomer, i.e., either cis- or trans-4F-GPB.

As used in this context, to “treat” means to ameliorate at least one symptom of the disorder. For example, administration of a therapeutically effective amount of a compound described herein for the treatment for neuropathic pain can result in a reduction in pain, or a reduction in requirement for other pain medications. Administration of a therapeutically effective amount of a compound described herein for the treatment of a condition associated with epilepsy, anxiety, or ataxia, will result in decreased frequency or severity of seizures, lessened anxiety, or lessened ataxia.

Pharmaceutical Compositions and Methods of Administration

The methods described herein include the use of pharmaceutical compositions comprising or consisting of a 4F-GBP as described herein as an active ingredient. In some embodiments, the compositions include the use of a mixture of the cis- and trans-4F-GPB isomers, or the compositions can be substantially pure (e.g., at least 80%, 85%, 90%, 95%, or 99%) or 100% pure for one isomer, i.e., either cis- or trans-4F-GPB.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions, e.g., pain medications.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### EXAMPLES

The invention is further described in the following examples, which do not limit the scope of the invention described in the claims.

### Example 1a. Methods of Synthesizing 4F-GBP

The NMR spectra were recorded on Bruker spectrometers AV400 (¹H, 300 MHz; ¹³C, 75 MHz) referenced to residual solvent signals as internal standards (¹H NMR: CDCl₃ 7.26 ppm and ¹³C{¹H} NMR: CDCl₃ 77.2 ppm). Concentrated solutions of samples in deuterated solvent were sealed off in a NMR tube for measurements. Commercially available reagents were purchased from SIGMA-Aldrich, Acros, Alfa-Assar or TCI and used as received.

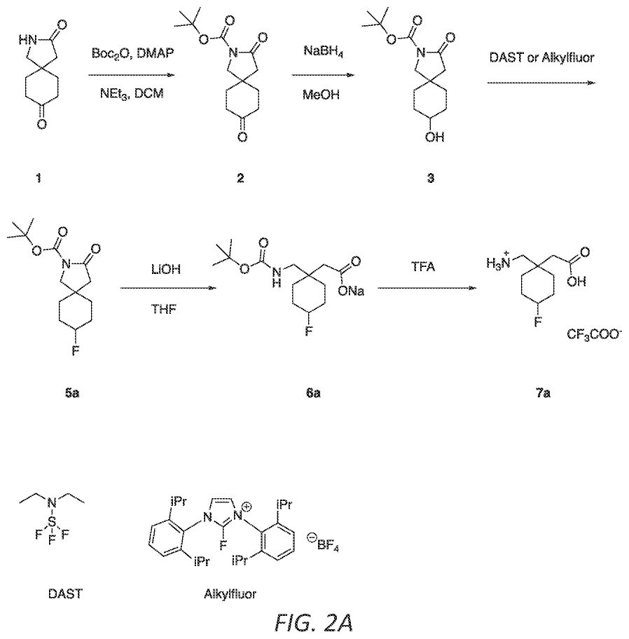

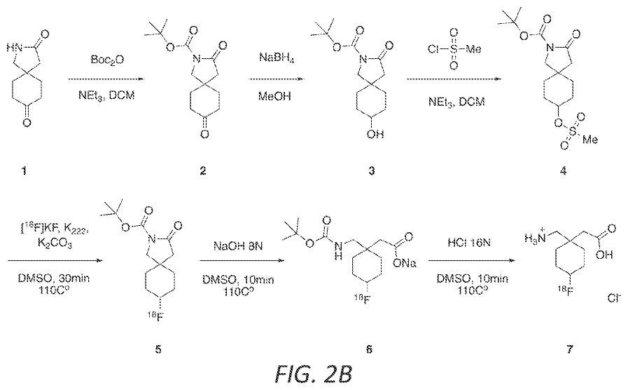

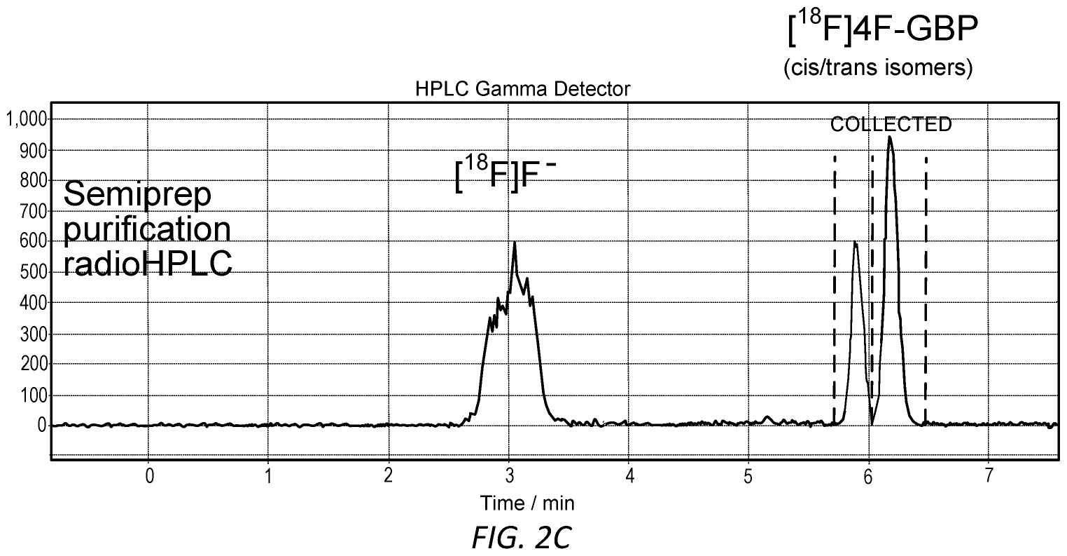

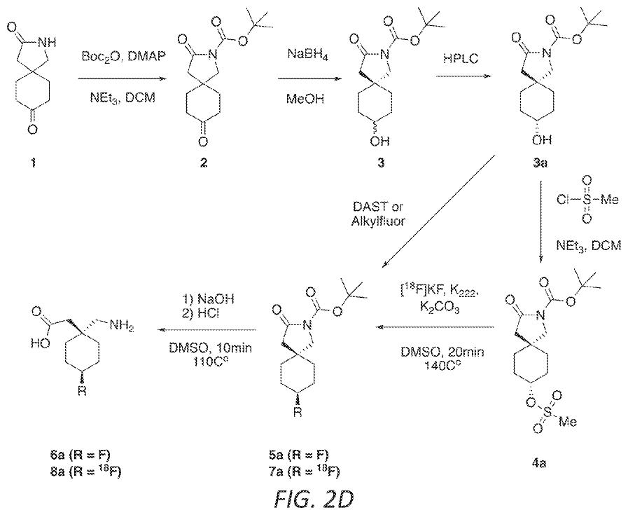

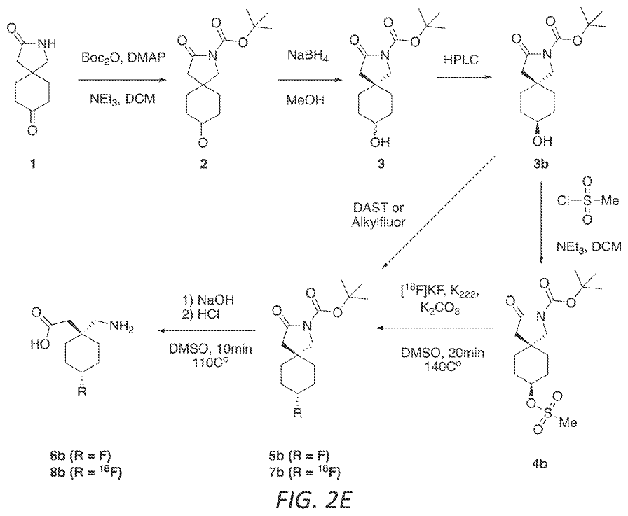

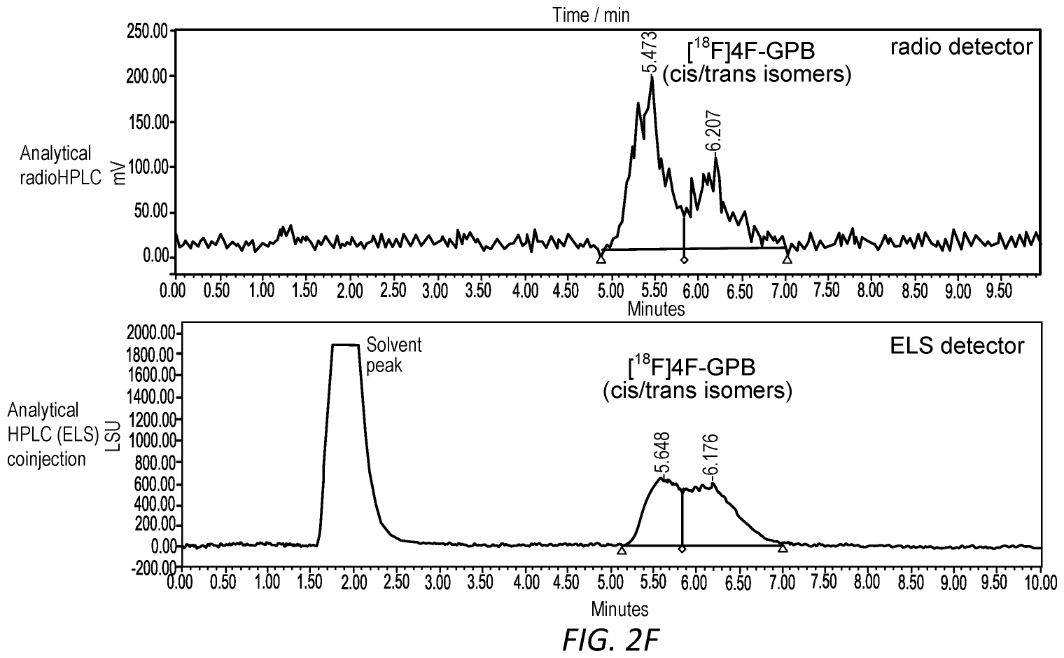

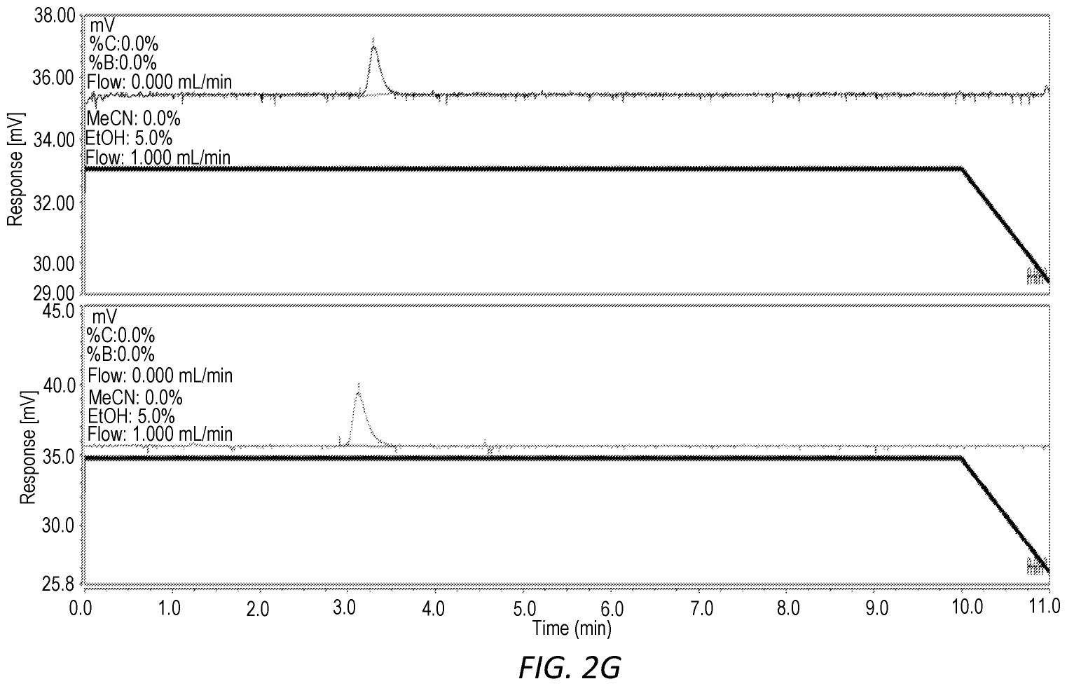

### 1.1. Syntheses and Characterizations of Cold Compounds (FIG. 2A)

2

To a 30 mL vial containing solid 1 (167 mg, 1 mmol), Di-tert-butyl dicarbonate (Boc₂O, 260 mg, 1.2 mmol), and 4-Dimethylaminopyridine (DMAP, 25 mg, 0.2 mmol), Dichloromethane (DCM, 10 mL) and triethylamine (NEt₃, 0.1 mL) were added via syringe. The mixture was stirred at room temperature for 2 hours. All volatiles were removed under reduced pressure. Residue was then washed with water and then extracted with DCM. Desired product (370 mg, 73% yield) was obtained after flash column as colorless oil. ¹H NMR (300 MHz, CDCl₃, 298 K): δ(ppm)=1.55 (s, 9H), 1.95 (t, J=6.7 Hz, 4H), 2.38-2.43 (m, 4H), 2.58 (s, 2H), 3.68 (s, 1H). ¹³C{¹H} NMR (75 MHz, CDCl₃, 298 K): δ(ppm)=28.01, 34.24, 35.65, 37.67, 44.07, 55.96, 83.40, 169.28, 171.93, 209.00.

3

To a 30 mL vial containing 2 (360 mg, 1.4 mmol) in 10 mL of methanol, NaBH₄ (120 mg, 3.2 mmol) was added in two portions. After stirring at room temperature for 2 hours, 1 mL of saturated NH₄Cl solution was added into the reaction mixture. Volatiles were then removed under reduced pressure. Residue was extracted with DCM and gave the desired product (350 mg, 97% yield) as colorless oil. ¹H NMR (300 MHz, CDCl₃, 298 K): δ(ppm)=1.42-1.48 (m, 4H), 1.52-1.53 (s×2, 9H), 1.73-1.87 (m, 4H), 2.36-2.43 (s×2, 2H), 3.47-3.55 (s×2, 2H), 3.48 (s, 1H), 3.71-3.76 (m, 1H). ¹³C{¹H}NMR (75 MHz, CDCl₃, 298 K): δ(ppm)=173.08, 172.95, 150.21, 82.94, 68.89, 68.28, 57.55, 55.84, 50.82, 45.76, 44.11, 34.24, 34.00, 33.29, 32.84, 31.20, 30.94, 28.44, 28.00.

5a

To a 30 mL vial containing 3 (25 mg, 0.1 mmol) in dichloromethane (DCM, 10 mL), diethylaminosulfur trifluoride (DAST, 40 mg, 0.25 mmol) was added via syringe at 0° C. dropwise, forming a yellow solution after stirring for 0.5 hour. Volatiles were removed under reduced pressure. Residue was dissolved in acetonitrile/water (1:1) mix solution and then purified by semi-prep HPLC affording colorless oil of the desired product (2 mg, 10% yield). ¹H NMR (300 MHz, CDCl₃, 298 K): δ(ppm)=1.47-1.53 (m, 11H), 1.76-1.96 (m, 6H), 2.93-2.41 (s×2, 2H), 3.52 (s×2, 2H), 4.60-4.78 (m, 1H). ¹³C{¹H} NMR (75 MHz, CDCl₃, 298 K): δ(ppm)=172.81, 172.62, 150.27, 150.13, 90.26, 89.86, 87.99, 87.60, 83.05, 83.02, 57.39, 55.89, 45.54, 43.95, 34.07, 33.99, 31.61, 31.52, 31.19, 31.12, 28.43, 28.36, 28.17, 28.01, 27.90, 27.75.

7a

To a 30 mL vial containing 5a (2 mg, 0.01 mmol) in tetrahydrofuran (THF, 1 mL), 0.1 mL of saturated LiOH solution was added via syringe dropwise. After stirring at room temperature for 3 hours, excess LiOH was quenched by adding 1 mL of saturated NH₄Cl solution into the reaction mixture. Intermediate 6a (6a was not purified and characterized) was obtained by extracting the mixture using ethyl acetate. Intermediate 6a was then transferred into a new 30 mL vial and 1 mL of trifluoroacetic acid (TFA) was added by syringe. After stirring at room temperature for 0.5 hour, clean solution was transfer into another 30 mL vial. Volatiles were removed under reduced pressure. Residue was extracted with 1 mL D₂O giving desired product as D₂O solution. ¹H NMR (300 MHz, D₂O, 298 K): δ(ppm)=1.31-1.76 (m, 8H), 2.39-2.41 (s×2, 2H), 2.96 (s, 2H), 3.65-3.80 (m, 1H). ¹³C{¹H} NMR (75 MHz, D₂O, 298 K): δ(ppm)=176.61, 91.49, 89.31, 47.20, 45.70, 39.10, 33.88, 28.81, 28.71, 27.65, 27.59, 26.23, 25.96, 25.83, 25.55.

### 1.2. Syntheses and Characterizations of Hot Compounds (FIG. 2B)

4

To a 30 mL vial containing 3 (25 mg, 0.1 mmol) and triethylamine (NEt₃, 0.05 mL) in dichloromethane (DCM, 10 mL), methanesulfonyl chloride (MsCl, 14 mg, 0.13 mmol) was added via syringe dropwise. The mixture was stirred at room temperature for 6 hours. All volatiles were removed under reduced pressure. Residue was then extracted with DCM. Desired product (25 mg, 71% yield) was obtained after flash column as colorless oil. ¹H NMR (300 MHz, CDCl₃, 298 K): δ(ppm)=1.47-1.58 (m, 11H), 1.75-1.91 (m, 6H), 2.38-2.42 (s×2, 2H), 3.02 (s, 3H), 3.50-3.54 (s×2, 2H), 4.74-4.80 (m, 1H). ¹³C{¹H} NMR (75 MHz, CDCl₃, 298 K): δ(ppm)=172.34, 150.11, 83.18, 83.15, 78.48, 56.50, 44.64, 38.78, 38.70, 33.85, 33.82, 32.35, 31.79, 29.12, 28.56, 28.39, 27.99.

### 1.3. Purification

The pure isomers were separated using semiprep HPLC (FIG. 2C), and purity and identity of the cold compounds was confirmed by HPLC and NMR. Identity of the radiolabeled compound was confirmed by coinjection with authentic standard on HPLC as well as coelution on TLC (visualized with ninhydrin stain) and radioTLC.

### 1.4. Alternative Trans-Isomer-Enriched Synthetic Method (FIGS. 2D-E)

The synthesis of 3a and 3b (see FIGS. 2D-E) from 2 was surprising as we were not expecting to be able to separate 3a and 3b using regular reverse-phase HPLC (non-chiral); standard methods would have used an stereoselective reduction reagent for step 2->3. One advantage of the present methods is that you get both isomers from the same reaction. Finally, for the last step (5a->6a and 7a->8a), it is preferred to use base first and then acid.

The cis/trans isomerism was assigned using 2D ¹H-NMR of 4a and 4b. Regular ¹H NMR of 5 and 6 was consistent with the assignment.

2

To a 30 mL vial containing solid 1 (167 mg, 1 mmol), Di-tert-butyl dicarbonate (Boc₂O, 260 mg, 1.2 mmol), and 4-Dimethylaminopyridine (DMAP, 25 mg, 0.2 mmol), Dichloromethane (DCM, 10 mL) and triethylamine (NEt₃, 0.1 mL) were added via syringe. The mixture was stirred at room temperature for 2 hours. All volatiles were removed under reduced pressure. Residue was then washed with water and then extracted with DCM. Desired product (370 mg, 73% yield) was obtained after flash column as colorless oil. 1H NMR (300 MHz, CDCl₃, 298 K): δ(ppm)=1.55 (s, 9H), 1.95 (t, J=6.7 Hz, 4H), 2.38-2.43 (m, 4H), 2.58 (s, 2H), 3.68 (s, 1H). 13C{¹H} NMR (75 MHz, CDCl₃, 298 K): δ(ppm)=28.01, 34.24, 35.65, 37.67, 44.07, 55.96, 83.40, 169.28, 171.93, 209.00.

3

To a 30 mL vial containing 2 (360 mg, 1.4 mmol) in 10 mL of methanol, NaBH₄ (120 mg, 3.2 mmol) was added in two portions. After stirring at room temperature for 2 hours, 1 mL of saturated NH₄Cl solution was added into the reaction mixture. Volatiles were then removed under reduced pressure. Residue was extracted with DCM and gave the desired product (350 mg, 97% yield) as colorless oil. ¹H NMR (300 MHz, CDCl₃, 298 K): δ(ppm)=1.42-1.48 (m, 4H), 1.52-1.53 (s×2, 9H), 1.73-1.87 (m, 4H), 2.36-2.43 (s×2, 2H), 3.47-3.55 (s×2, 2H), 3.48 (s, 1H), 3.71-3.76 (m, 1H). ¹³C{¹H}NMR (75 MHz, CDCl₃, 298 K): δ(ppm)=173.08, 172.95, 150.21, 82.94, 68.89, 68.28, 57.55, 55.84, 50.82, 45.76, 44.11, 34.24, 34.00, 33.29, 32.84, 31.20, 30.94, 28.44, 28.00.

3a

Compound 3 was dissolved in acetonitrile/water (1:1) mix solution and then purified by semi-prep HPLC giving colorless oil of the desired product. ¹H NMR (300 MHz, CDCl₃, 298 K): δ(ppm)=1.33-1.97 (m, 17H), 2.36 (s, 2H), 3.55 (s, 2H), 3.72-3.78 (m, 1H). ¹³C{¹H} NMR (75 MHz, CDCl₃, 298 K): δ(ppm)=173.28, 150.39, 83.15, 68.47, 56.02, 45.96, 34.18, 33.04, 31.13, 28.63, 28.20.

3b

Compound 3 was dissolved in acetonitrile/water (1:1) mix solution and then purified by semi-prep HPLC giving colorless oil of the desired product. ¹H NMR (300 MHz, CDCl₃, 298 K): δ(ppm)=1.3-1.87 (m, 17H), 2.42 (s, 2H), 3.47 (s, 2H), 3.65-3.75 (m, 1H). ¹³C{¹H} NMR (75 MHz, CDCl₃, 298 K): δ(ppm)=173.15, 150.40, 83.12, 69.07, 57.75, 44.30, 34.43, 33.48, 31.37, 28.61, 28.18.

4a

To a 30 mL vial containing 3a (25 mg, 0.1 mmol) and triethylamine (NEt₃, 0.05 mL) in dichloromethane (DCM, 10 mL), methanesulfonyl chloride (MsCl, 14 mg, 0.13 mmol) was added via syringe dropwise. The mixture was stirred at room temperature for 6 hours. All volatiles were removed under reduced pressure. Residue was then extracted with DCM. Desired product (25 mg, 71% yield) was obtained after flash column as colorless oil. ¹H NMR (300 MHz, CDCl₃, 298 K): δ(ppm)=1.48-1.57 (m, 11H), 1.74-1.91 (m, 6H), 2.9 (s, 2H), 3.02 (s, 3H), 3.55 (s, 2H), 4.77-4.84 (m, 1H). ¹³C{¹H}NMR (75 MHz, CDCl₃, 298 K): δ(ppm)=172.61, 150.26, 83.35, 78.52, 56.69, 44.82, 38.90, 34.02, 31.99, 28.59, 28.20.

4b

To a 30 mL vial containing 3b and triethylamine (NEt₃, 0.05 mL) in dichloromethane (DCM, 10 mL), methanesulfonyl chloride (MsCl, 14 mg, 0.13 mmol) was added via syringe dropwise. The mixture was stirred at room temperature for 6 hours. All volatiles were removed under reduced pressure. Residue was then extracted with DCM. Desired product was obtained after flash column as colorless oil. ¹H NMR (300 MHz, CDCl₃, 298 K): δ(ppm)=1.47-1.57 (m, 11H), 1.73-1.84 (m, 4H), 1.93-2.01 (m, 2H), 2.43 (s, 2H), 3.50 (s, 3H), 3.55 (s, 2H), 4.74-4.79 (m, 1H). ¹³C{¹H} NMR (75 MHz, CDCl₃, 298 K): δ(ppm)=172.31, 150.12, 83.19, 78.47, 38.78, 33.85, 32.35, 28.57, 28.01, 28.00.

5a

To a 30 mL vial containing 3a in dichloromethane (DCM, 10 mL), diethylaminosulfur trifluoride (DAST) was added via syringe at 0° C. dropwise, forming a yellow solution after stirring for 0.5 hour. Volatiles were removed under reduced pressure. Residue was dissolved in acetonitrile/water (1:1) mix solution and then purified by semi-prep HPLC affording colorless oil of the desired product. ¹H NMR (300 MHz, CDCl₃, 298 K): δ(ppm)=1.47-1.58 (m, 11H), 1.73-1.88 (m, 6H), 2.41 (s, 2H), 3.52 (s, 2H), 4.55-4.78 (m, 1H). ¹³C{¹H} NMR (75 MHz, CDCl₃, 298 K): δ(ppm)=172.82, 150.46, 90.46, 88.19, 83.24, 56.05, 45.73, 34.18, 34.17, 31.80, 31.71, 28.36, 28.20, 28.09. ¹⁹F NMR (282 MHz, CDCl₃, 298 K): δ(ppm)=−176.34.

5b

To a 30 mL vial containing 3a in dichloromethane (DCM, 10 mL), diethylaminosulfur trifluoride (DAST) was added via syringe at 0° C. dropwise, forming a yellow solution after stirring for 0.5 hour. Volatiles were removed under reduced pressure. Residue was dissolved in acetonitrile/water (1:1) mix solution and then purified by semi-prep HPLC affording colorless oil of the desired product. ¹H NMR (300 MHz, CDCl₃, 298 K): δ(ppm)=1.46-1.53 (m, 11H), 1.66-1.90 (m, 6H), 2.39 (s, 2H), 3.52 (s, 2H), 4.59-4.80 (m, 1H). ¹³C{¹H} NMR (75 MHz, CDCl₃, 298 K): δ(ppm)=172.81, 150.12, 89.87, 87.61, 83.01, 57.38, 43.96, 34.08, 34.07, 31.19, 31.12, 28.43, 28.02, 28.00, 27.75. ¹⁹F NMR (282 MHz, CDCl₃, 298 K): δ(ppm)=−176.73.

6a (trans-GBP4F or GBP4F iso2)

To a 30 mL vial containing 5a (2 mg, 0.01 mmol) in tetrahydrofuran (THF, 1 mL), 0.1 mL of saturated LiOH solution was added via syringe dropwise. After stirring at room temperature for 3 hours, excess LiOH was quenched by adding 1 mL of saturated NH₄Cl solution into the reaction mixture. Intermediate was obtained by extracting the mixture using ethyl acetate. Intermediate was then transferred into a new 30 mL vial and 1 mL of trifluoroacetic acid (TFA) was added by syringe. After stirring at room temperature for 0.5 hour, clean solution was transfer into another 30 mL vial. Volatiles were removed under reduced pressure. Residue was extracted with H₂O and then purified by semi-prep HPLC giving desired product. ¹H NMR (300 MHz, D₂O, 298 K): δ(ppm)=1.39-1.48 (m, 2H), 1.69-1.96 (m, 6H), 2.48 (s, 2H), 3.04 (s, 2H), 4.66-4.90 (m, 1H). ¹³C{¹H} NMR (75 MHz, D₂O, 298 K): δ(ppm)=180.11, 92.79, 90.61, 47.24, 45.63, 33.53, 29.30, 29.20, 26.51, 26.25. ¹⁹F NMR (282 MHz, D₂O, 298 K): δ(ppm)=−171.12.

6b (cis-GBP4F or GBP4F iso1)

To a 30 mL vial containing 5b (2 mg, 0.01 mmol) in tetrahydrofuran (THF, 1 mL), 0.1 mL of saturated LiOH solution was added via syringe dropwise. After stirring at room temperature for 3 hours, excess LiOH was quenched by adding 1 mL of saturated NH₄Cl solution into the reaction mixture. Intermediate was obtained by extracting the mixture using ethyl acetate. Intermediate was then transferred into a new 30 mL vial and 1 mL of trifluoroacetic acid (TFA) was added by syringe. After stirring at room temperature for 0.5 hour, clean solution was transfer into another 30 mL vial. Volatiles were removed under reduced pressure. Residue was extracted with H₂O and then purified by semi-prep HPLC giving desired product. ¹H NMR (300 MHz, D₂O, 298 K): δ(ppm)=1.46-1.54 (m, 2H), 1.59-1.68 (m, 2H), 1.79-1.92 (m, 4H), 2.46 (s, 2H), 3.05 (s, 2H), 4.71-5.00 (m, 1H). ¹³C{¹H} NMR (75 MHz, D₂O, 298 K): δ(ppm)=180.26, 91.95, 89.78, 48.46, 44.03, 33.56, 28.18, 28.12, 26.10, 25.83. ¹⁹F NMR (282 MHz, D₂O, 298 K): δ(ppm)=−174.80.

8a (trans/iso2) and 8b (cis/iso1):

[¹⁸F]KF is made by bombardment of a H₂¹⁸O with high energy protons in cyclotron. [¹⁸F]KF is loaded onto a QMA cartridge and then eluted with kryptofix-2.2.2. (K₂₂₂)/K₂CO₃ water/acetonitrile solution (1:1). The mixture is dried under nitrogen flow and then ˜0.5 mg of precursor 4a or 4b dissolved in 0.3 mL DMSO is added. The ¹⁸F labeled Boc protected gabapentin lactam (5a or 5b) is obtained by the reaction [¹⁸F]KF and 4a or 4b in the presence of kryptofix-2.2.2. (K₂₂₂)/K₂CO₃ at 140 C.° for 20 min. ¹⁸F-labeled gabapentin 8a (iso2) or 8b (iso1) is obtained by the hydrolysis and deprotection of 5a or 5b using NaOH (2N) and HCl (3N), respectively. Reversed-phase HPLC is used for the purification of 8a (iso2) or 8b (iso1) using XBridge 5 um C18 semiprep-column (10×250 mm). The mobile phase is NaH₂PO₄ EtOH-H₂O (10 mM, v/v=5:95) solution at flow rate of 4 mL/min.

### Example 1b. Methods of Synthesizing 4F-Gabapentin Lactam

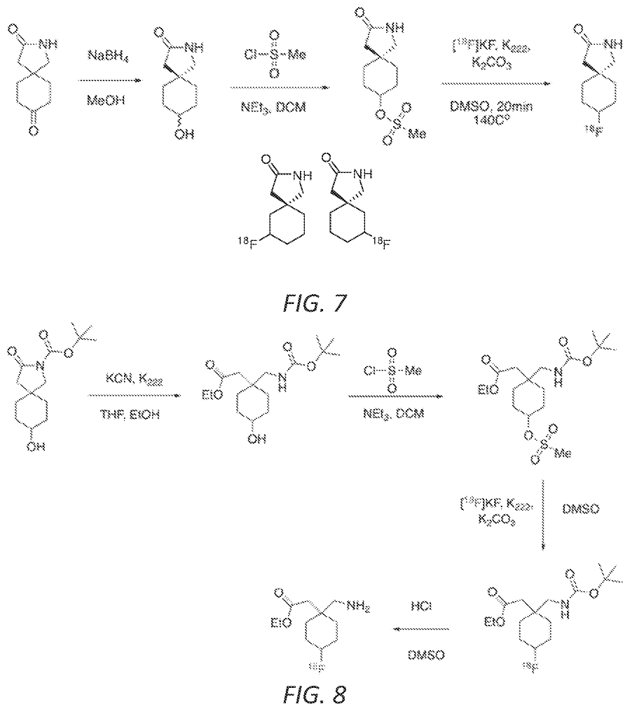

FIG. 7 shows exemplary synthesis of a 4[¹⁸F]-gabapentin lactam compound within Formula (II). Noteworthy, these compounds may be useful as intermediates in the synthesis of 4-fluorogabapentin compounds of Formula (I), as shown, for examples, in FIGS. 2A (compound 5a), 2B (compound 5), 2D (compounds 5a and 7a), and 2E (compounds 5b and 7b). Similar methods can be utilized to prepare 3F-, and 4F-gabapentin lactam compounds (see FIG. 7, bottom row).

### Example 1c. Synthesis of [¹⁸F]4-fluorogabapentin ethyl ester

The synthesis is shown in FIG. 8. Starting from a hydroxyl starting material compound, the hydrolysis of hydroxyl lactam in the presence of ethanol gave tert-butyloxycarbonyl (Boc) protected hydroxyl ester. The ester was further reacted with methanesulfonyl chloride (MsCl) to generate the corresponding mesylate compound. The corresponding ¹⁸F-labeled Boc protected gabapentin ethyl ester was then obtained by the reaction with [¹⁸F]KF in the presence of kryptofix-2.2.2. (K₂₂₂)/K₂CO₃ at high temperature. ¹⁸F-labeled gabapentin ethyl ester was obtained by the removal of the Boc protecting group with hydrochloric acid. The nonradioactive version of this compound was synthesized by using a non-radioactive fluoride source.

Additional esters were synthesized as described for [¹⁸F]4-fluorogabapentin ethyl ester using the corresponding hydroxyl derivative (OH in 7 or 9 position) and other alcohols for esterification including methanol, propanol, isopropanol, butanol and isobutanol.

### Example 1d. Synthesis Methods of [¹⁸F]4-fluorogabapentin Prodrug (Amine Protected, Carbamate)

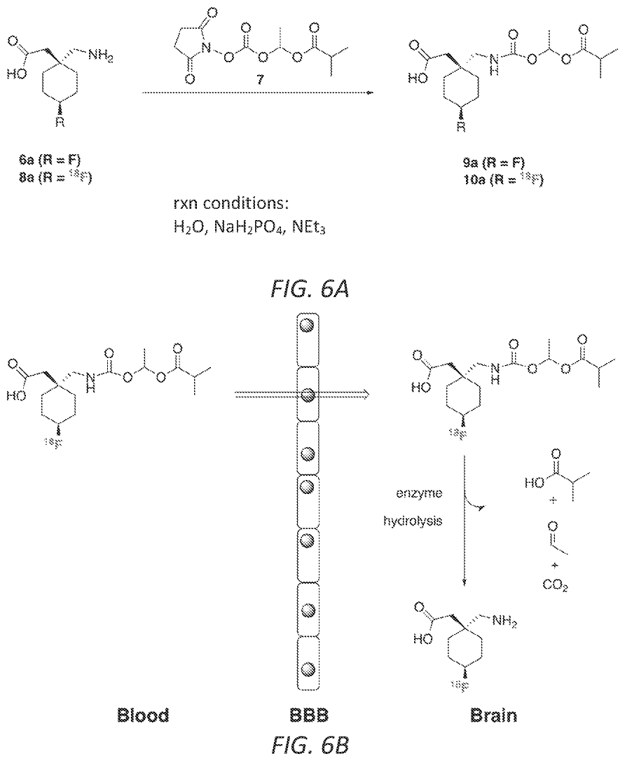

The synthesis started from corresponding ¹⁸F-labeled gabapentin, as shown in FIG. 6A. The corresponding ¹⁸F-labeled gabapentin prodrug was obtained by the direct reaction of ¹⁸F-labeled gabapentin with the N-hydroxysuccinimide ester shown in FIG. 6A. Other carbamates can be generated by using different N-hydroxysuccinimide esters.

### Example 1e. Synthesis of 3-fluorogabapentin and [¹⁸F]3-fluorogabapentin

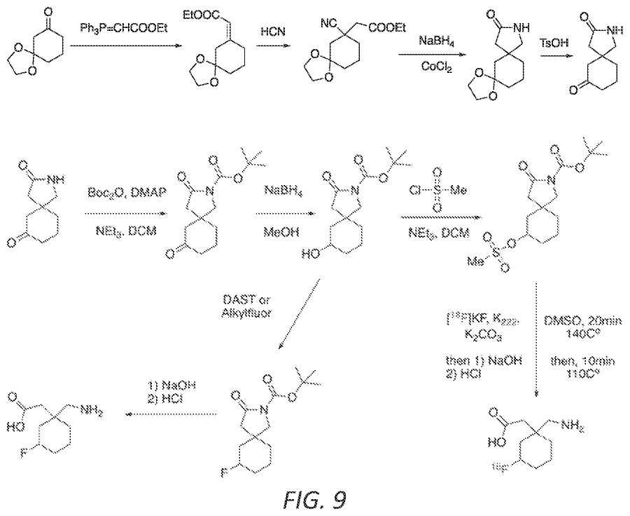

2-azaspiro[4.5]decane-3,7-dione is synthesized as shown in FIG. 9. Namely, 1,4-dioxaspiro[4.5]decan-7-one is reacted with (carbethoxymethylene)triphenylphosphorane followed by cyanation and reduction with sodium borohydride in the presence of cobalt (II) chloride. The resultant product was deprotected with tosylic acid to generate the corresponding 2-azaspiro[4.5]decane-3,7-dione. 3-fluorogabpentin from this precursor is synthesized according to FIG. 9 following the procedure detailed for 4-fluorogabapentin. The corresponding ¹⁸F-labeled version was synthesized according to FIG. 9 following the procedure described for [¹⁸F]4-fluorogabapentin in the previous example.

### Example 1f. Synthesis of 5-fluorogabapentin and [¹⁸F]5-fluorogabapentin

[5F]-gabapentin compounds were synthesized following the same procedure as [3F]-gabapentin and [¹⁸F]3-fluorogabapentin from 2-azaspiro[4.5]decane-3,9-dione.

### Example 2. Radiolabeled ¹⁸F-fluorogabapentin Binds to α2δ-1 Receptors in Mice and Rats

As noted above, the expression of α2δ-1 is increased in areas involved in nociception (nerves, DRGs and spinal cord). In order to assess the potential of using radiolabeled gabapentin to measure changes in α2δ-1 expression, several experiments were performed, as follows.

microPET/CT: mice and rats were anesthetized with isoflurane and positioned in the bed of a microPET/CT instrument. 100-400 μCi of [¹⁸F]4F-GBP isomer 2 were injected through a tail vein catheter. Animals were imaged using dynamic PET from 0 to 120 min to provide greatest contrast. Specific binding was assessed by coinjection with pharmacological doses of nonradiolabeled gabapentin (100 mg/kg) or preinjection of pregabalin (25 mg/kg). Rodents were fasted from the night before to maximize tracer uptake as reported for melphalan, a drug that is transported by the same L-type amino acid transporter (LAT-1).

Biodistribution: Mice were euthanized and their tissues dissected 10, 30, 60 and 120 min after injection of [¹⁸F]F-GBP and their major organs dissected and counted. Rats were euthanized 120 min after tracer injection and their brain, muscle and spinal cord dissected for gamma counting, autorad and IHC.

Data analysis: PET images were reconstructed using the scanner software and a rat brain atlas automatically fitted to the data using VivoQuant. Time activity curves (TACs) were extracted for multiple brain regions and spinal cord segments (lumbar, thoracic and cervical cord). We used the brain TACs from naïve rats to assess test/retest variability and specific binding. We compared tracer binding in the spinal cord and brain of SNL vs. sham. The investigator performing the analysis was blinded to the animal type (sham or SNL).

IHC: we stained for α2δ-1 in the cord following the procedures published by Taylor and Garrido⁴⁵.

Autoradiography: Rat brains and mouse spinal cords from a mouse model of neuropathic pain (spared nerve injury) were used in this study. Tissues from mice with neuropathic pain were used (Spared nerve injury model, Decosterd and Woolf, Pain. 2000 August; 87(2):149-158). For autoradiography, 20 m thick sections were incubated with 10 nCi of [³H]gabapentin or [¹⁸F]4F-GBP in PBS for 30 min in the presence or absence of cold competitors (5 μM gabapentin, pregabalin or 4-fluorogabapentin) and then washed with ice cold PBS (3×1 min). The slides were dried under a stream of nitrogen and apposed onto an imaging plate overnight ([¹⁸F]4F-GBP) or 14 days ([³H]gabapentin). Afterwards, the plate was digitized using a Typhoon 9000 phosphorimager.

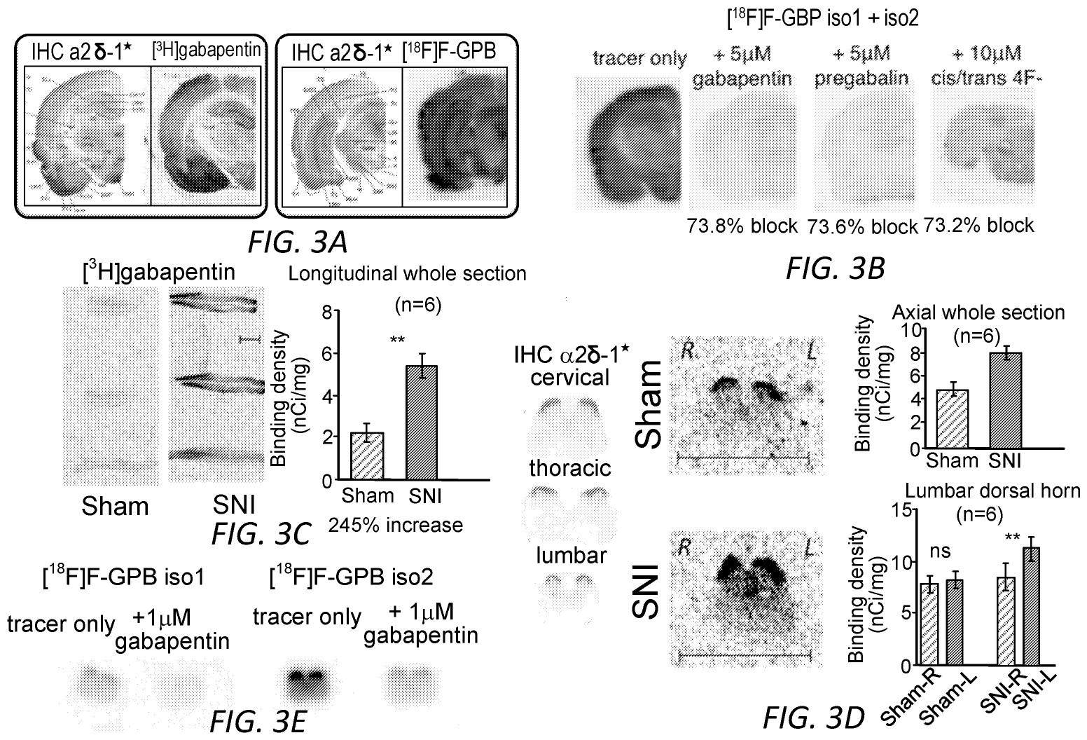

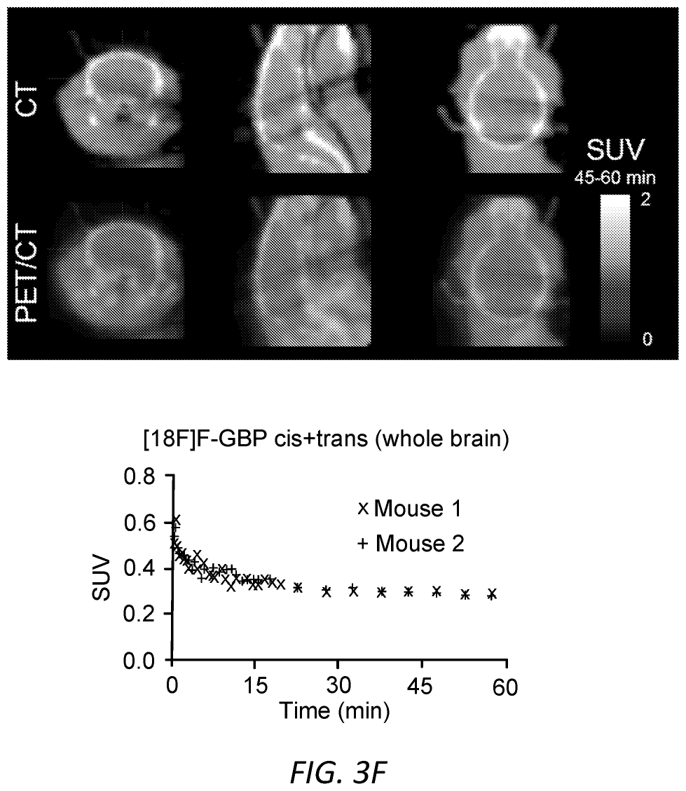

In the first experiment, we performed a qualitative comparison of the distribution of the binding of [³H]gabapentin and cis/trans [¹⁸F]4F-GBP with the published distribution of α2δ-1 in the rat brain (FIG. 3A). [³H]gabapentin was chosen because it is commercially available and because the low energy of the β-particles provides higher autoradiographic resolution than fluorine-18. This experiment showed very similar distribution of α2δ-1 immunostaining, [³H]gabapentin and cis/trans [¹⁸F]4F-GBP in the rat brain²⁵. In the second experiment, we confirmed the specific binding of cis/trans [¹⁸F]4F-GBP in rat brain sections by blocking with gabapentin, pregabalin and cis/trans 4-fluorogabapentin (FIG. 3B). In the next experiment, we examined the binding of [³H]gabapentin in spinal cord sections from mice with neuropathic pain. This experiment showed a ˜245% increase in whole longitudinal sections (FIG. 3C) and a ˜165% increase in binding in whole axial sections (FIG. 3D), demonstrating that it is possible to detect changes in neuropathic pain models using radioactive versions of GBP. Detailed examination of the axial sections showed a distribution pattern consistent with α2δ-1 immunostaining and a 38% increase in binding on the affected side (ipsilateral to the injury) compared to the contralateral side. Finally, we performed autoradiography with the pure isomers of [¹⁸F]4F-GBP and observed that trans-4F-GBP was more potent than cis-4F-GBP (FIG. 3E); both compounds get into the brain (FIG. 3F). In summary, these experiments prove that [¹⁸F]4F-GBP binds to α2δ-1 receptors, that trans-4F-GBP was more potent and that the binding of radiolabeled gabapentin was higher in the spinal cords of mice with neuropathic pain than control.

### Example 3. Evaluation of [¹⁸F]-4F-GBP in Rats with Neuropathic Pain

We evaluated uptake of [¹⁸F]4F-GBP in the L4-L6 spinal nerves in a neuropathic pain model (spinal nerve ligation model) 75 min after in vivo administration. In this rat model, the L5 and L6 spinal nerves on the right side are tightly ligated. This surgery leads to a neuropathic pain phenotype. The animals develop allodynia and hyperalgesia 3 to 7 days-post-surgery which persists for one month or longer. As noted above, previous investigators have shown that there is massive upregulation of α2δ-1 receptor in the injured nerve side.

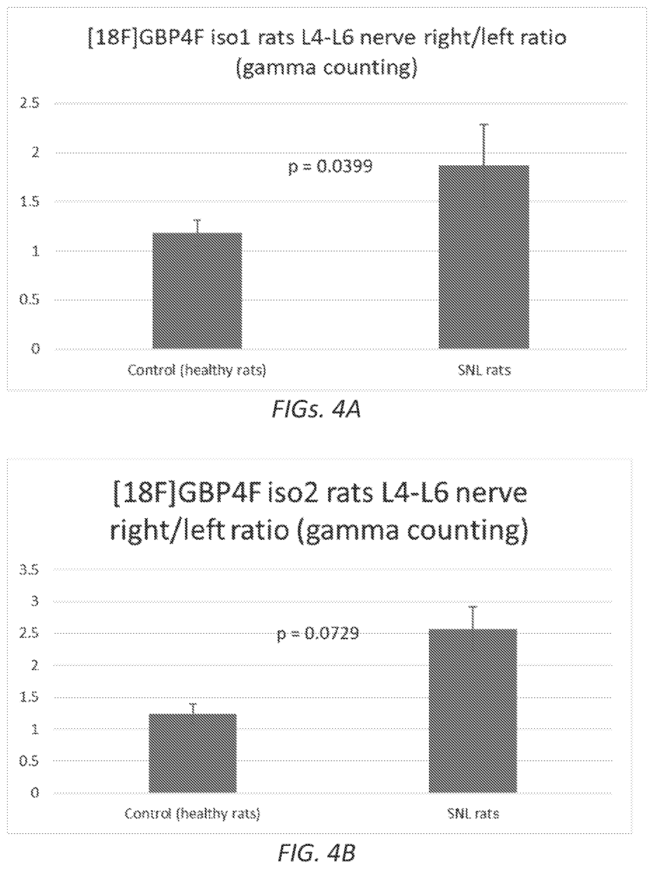

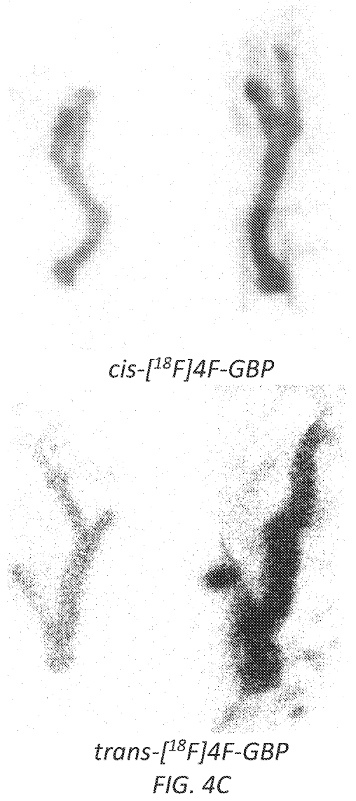

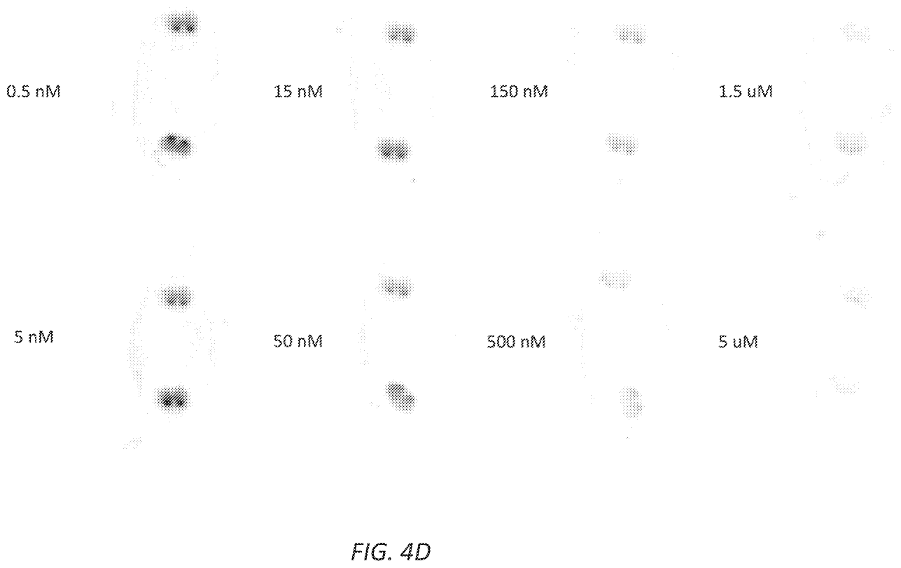

SNL and Sham animals were acquired from Charles River. We scanned the rats longitudinally. 1-2 weeks after the surgery we administered the radiolabeled compound and measured the uptake of the radioactivity in the injured nerve (right side) vs. uninjured nerve (left side) using gamma counting. The results, presented in FIGS. 4A-B, showed increased binding in the injured nerve, supporting use for detection of neuropathic pain in vivo using PET. Autoradiography of L4-L6 explanted spinal nerves from the neuropathic rat model after in vivo administration of [¹⁸F]4F-GBP showed that the injured right nerve (right side) had significantly higher binding of both cis- and trans-[-F]4F-GBP than the left nerve (left side), see FIG. 4C.

In vitro autoradiography of spinal cord sections was used to quantify the binding potency of 4F-GBP derivatives and gabapentin. The results, seen in FIG. 4D, showed that trans-[¹⁸F]4F-GBP had higher binding affinity than gabapentin whereas cis-[¹⁸F]4F-GBP4 had lower binding affinity. We calculated the EC50 of the trans isomer at 91.9 nM when blocked with the cold 4F-GBP4, and 132.6 nM when blocked with GBP. For the cis isomer, calculated the EC50 of the trans isomer at 3.3 uM when blocked with the cold 4F-GBP4, and 196 nM when blocked with GBP

### Example 4. PET Imaging in Nonhuman Primates (NIPs) with ¹⁸F-GBP

NHPs are ideal for characterizing novel PET radioligands given their large brain size and similar metabolism to humans.

General imaging procedure: The rhesus monkey was sedated by intramuscular injection of ketamine in its housing facility. Following sedation, the animal was intubated and placed inside a primate transport cart. The animal was transported to the scanner room and laid on the scanner bed in supine position. The tube was connected to an isoflurane evaporator and anesthesia induced and maintained with 1-1.5% isoflurane in O₂. Vital signs including heart rate, respiratory rate, SpO₂, exhaled CO₂, blood pressure and temperature were continuously monitored for the duration of the scan. One intravenous catheter was placed in the saphenous vein for administration of the tracer. One intraarterial catheter was placed in the posterior tibial artery for blood sampling. Following a low dose CT, the animal received ˜5 mCi of [¹⁸F]4F-GBP in 10 mL of saline intravenously (3 min bolus injection) and a dynamic PET scan was performed for up to 3 h. Serial arterial blood samples were collected during the scan and processed to measure radioactivity concentration in whole blood and plasma. Selected plasma samples were analyzed for radiometabolites using radioHPLC. MRI was coregistered to the PET/CT and TACs extracted for each brain region defined on an MR atlas.

Study design: We scanned two male rhesus macaques. Each monkey was scanned two times. In the first session, we performed 3h dynamic scans (baseline) of each monkey. On the second session, the monkey underwent a blocking scan with coinjection of gabapentin (5 mg/kg).

Data analysis: Pharmacokinetic modeling using metabolite-corrected arterial input function was performed. Volume of distribution (VT), binding potential (BP) other parameters were calculated.

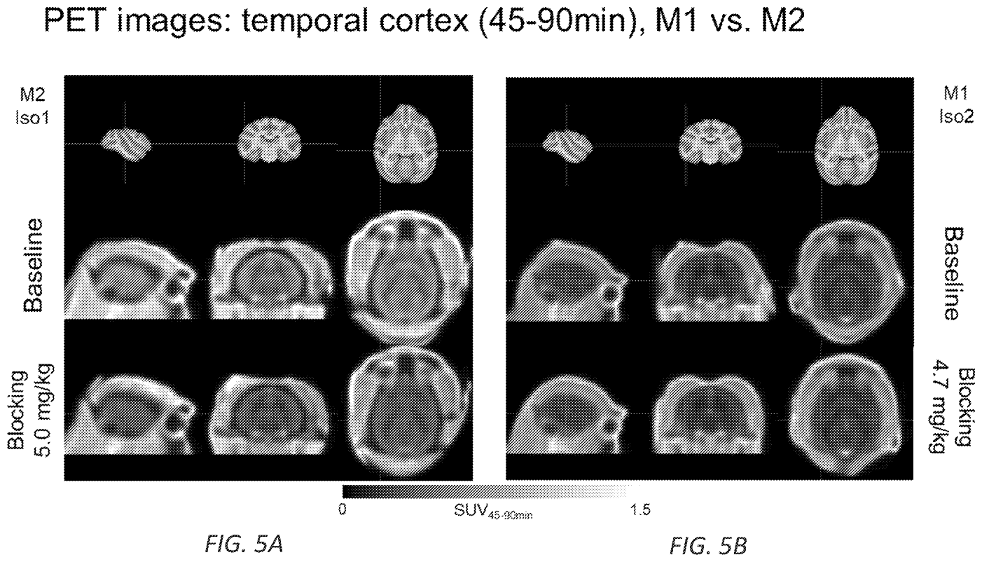

The results, seen in FIGS. 5A-B, showed significant brain penetration for both the cis (5A) and trans (5B) isomers. At the concentration tested, the cis-[¹⁸F]4F-GBP isomer showed clear specific (displaceable) binding.

### REFERENCES

1. Luo, Z. D. et al. Upregulation of dorsal root ganglion (alpha)2(delta) calcium channel subunit and its correlation with allodynia in spinal nerve-injured rats. The Journal of neuroscience:the official journal of the Society for Neuroscience 21, 1868-1875 (2001).
2. Newton, R. A., Bingham, S., Case, P. C., Sanger, G. J. & Lawson, S. N. Dorsal root ganglion neurons show increased expression of the calcium channel alpha2delta-1 subunit following partial sciatic nerve injury. Brain Res Mol Brain Res 95, 1-8 (2001).
3. Bauer, C. S. et al. The increased trafficking of the calcium channel subunit alpha2delta-1 to presynaptic terminals in neuropathic pain is inhibited by the alpha2delta ligand pregabalin. The Journal of neuroscience:the official journal of the Society for Neuroscience 29, 4076-4088, (2009).
4. Nieto-Rostro, M. et al. Altered expression of the voltage-gated calcium channel subunit alpha(2)delta-1: a comparison between two experimental models of epilepsy and a sensory nerve ligation model of neuropathic pain. Neuroscience 283, 124-137, (2014).
5. Vollmer, K. O., von Hodenberg, A. & Kolle, E. U. Pharmacokinetics and metabolism of gabapentin in rat, dog and man. Arzneimittelforschung 36, 830-839 (1986).
6. Calandre, E. P., Rico-Villademoros, F. & Slim, M. Alpha2delta ligands, gabapentin, pregabalin and mirogabalin: a review of their clinical pharmacology and therapeutic use. Expert Rev Neurother 16, 1263-1277, (2016).
7. Luo, Z. D. et al. Injury type-specific calcium channel alpha 2 delta-1 subunit up-regulation in rat neuropathic pain models correlates with antiallodynic effects of gabapentin. The Journal of pharmacology and experimental therapeutics 303, 1199-1205 (2002).
8. Li, K. W. et al. Calcium channel alpha2delta1 proteins mediate trigeminal neuropathic pain states associated with aberrant excitatory synaptogenesis. The Journal of biological chemistry 289, 7025-7037 (2014).
9. Zhang, X., Bai, X. & Zhang, Q. [Change of calcium channel alpha-2-delta-1 subunit expression levels in trigeminal neuralgia rats]. Hua Xi Kou Qiang Yi Xue Za Zhi 30, 314-316 (2012).
10. Kusuyama, K. et al. Upregulation of calcium channel alpha-2-delta-1 subunit in dorsal horn contributes to spinal cord injury-induced tactile allodynia. Spine J 18, 1062-1069 (2018).
11. Boroujerdi, A. et al. Calcium channel alpha-2-delta-1 protein upregulation in dorsal spinal cord mediates spinal cord injury-induced neuropathic pain states.

Pain 152, 649-655 (2011).
12. Xiao, W., Boroujerdi, A., Bennett, G. J. & Luo, Z. D. Chemotherapy-evoked painful peripheral neuropathy: analgesic effects of gabapentin and effects on expression of the alpha-2-delta type-1 calcium channel subunit. Neuroscience 144, 714-720 (2007).
13. Chen, Y., Chen, S. R., Chen, H., Zhang, J. & Pan, H. L. Increased alpha2delta-1-NMDA receptor coupling potentiates glutamatergic input to spinal dorsal horn neurons in chemotherapy-induced neuropathic pain. Journal of neurochemistry 148, 252-274 (2019).
14. Brugarolas, P. et al. Development of a PET radioligand for potassium channels to image CNS demyelination. Sci Rep 8, 607 (2018).
15. Brugarolas, P., Freifelder, R., Cheng, S. H. & DeJesus, O. Synthesis of meta-substituted [18F]3-fluoro-4-aminopyridine via direct radiofluorination of pyridine N-oxides. Chem Commun (Camb) 52, 7150-7152 (2016).
16. Brugarolas, P., Bhuiyan, M., Kucharski, A. & Freifelder, R. Automated Radiochemical Synthesis of [18F]3F4AP: A Novel PET Tracer for Imaging Demyelinating Diseases. J Vis Exp, doi:10.3791/55537 (2017).
17. Basuli, F., Zhang, X., Brugarolas, P., Reich, D. S. & Swenson, R. E. An efficient new method for the synthesis of 3-[18F]fluoro-4-aminopyridine via Yamada-Curtius rearrangement. J Labelled Comp Radiopharm 61, 112-117 (2018).
18. Brugarolas, P., Reich, D. S. & Popko, B. Detecting Demyelination by PET: The Lesion as Imaging Target. Mol Imaging 17, 1536012118785471 (2018).
19. Catterall, W. A. Voltage-gated calcium channels. Cold Spring Harb Perspect Biol 3, a003947 (2011).
20. Li, C. Y. et al. Calcium channel alpha2delta1 subunit mediates spinal hyperexcitability in pain modulation. Pain 125, 20-34 (2006).
21. Taylor, C. P. Mechanisms of action of gabapentin. Rev Neurol (Paris) 153 Suppl 1, S39-45 (1997).
22. Gee, N. S. et al. The novel anticonvulsant drug, gabapentin (Neurontin), binds to the alpha2delta subunit of a calcium channel. The Journal of biological chemistry 271, 5768-5776 (1996).
23. Bryans, J. S. et al. Identification of novel ligands for the gabapentin binding site on the alpha2delta subunit of a calcium channel and their evaluation as anticonvulsant agents. Journal of medicinal chemistry 41, 1838-1845, doi:10.1021/jm970649n (1998).
24. Marais, E., Klugbauer, N. & Hofmann, F. Calcium channel alpha(2)delta subunits-structure and Gabapentin binding. Mol Pharmacol 59, 1243-1248 (2001).
25. Hill, D. R., Suman-Chauhan, N. & Woodruff, G. N. Localization of [3H]gabapentin to a novel site in rat brain: autoradiographic studies. Eur J Pharmacol 244, 303-309 (1993).
26. Li, Z. et al. Pregabalin is a potent and selective ligand for alpha(2)delta-1 and alpha(2)delta-2 calcium channel subunits. Eur J Pharmacol 667, 80-90 (2011).
27. Field, M. J. et al. Identification of the alpha2-delta-1 subunit of voltage-dependent calcium channels as a molecular target for pain mediating the analgesic actions of pregabalin. Proceedings of the National Academy of Sciences of the United States of America 103, 17537-17542 (2006).
28. Lotarski, S. M. et al. Anxiolytic-like activity of pregabalin in the Vogel conflict test in alpha2delta-1 (R217A) and alpha2delta-2 (R279A) mouse mutants. The Journal of pharmacology and experimental therapeutics 338, 615-621 (2011).
29. Hendrich, J. et al. Pharmacological disruption of calcium channel trafficking by the alpha2delta ligand gabapentin. Proceedings of the National Academy of Sciences of the United States of America 105, 3628-3633 (2008).
30. Gilron, I., Watson, C. P., Cahill, C. M. & Moulin, D. E. Neuropathic pain: a practical guide for the clinician. CMAJ 175, 265-275 (2006).
31. Verma, V., Singh, N. & Singh Jaggi, A. Pregabalin in neuropathic pain: evidences and possible mechanisms. Curr Neuropharmacol 12, 44-56 (2014).
32. Moore, A., Derry, S. & Wiffen, P. Gabapentin for Chronic Neuropathic Pain. JAMA 319, 818-819 (2018).
33. Cheshire, W. P., Jr. Defining the role for gabapentin in the treatment of trigeminal neuralgia: a retrospective study. J Pain 3, 137-142 (2002).
34. Stefani, A., Spadoni, F. & Bernardi, G. Gabapentin inhibits calcium currents in isolated rat brain neurons. Neuropharmacology 37, 83-91 (1998).
35. Meder, W. P. & Dooley, D. J. Modulation of K+-induced synaptosomal calcium influx by gabapentin. Brain research 875, 157-159 (2000).
36. Yu, Y. P., Gong, N., Kweon, T. D., Vo, B. & Luo, Z. D. Gabapentin prevents synaptogenesis between sensory and spinal cord neurons induced by thrombospondin-4 acting on pre-synaptic Cav alpha2 delta1 subunits and involving T-type Ca2+ channels. British journal of pharmacology 175, 2348-2361 (2018).
37. Eroglu, C. et al. Gabapentin receptor alpha2delta-1 is a neuronal thrombospondin receptor responsible for excitatory CNS synaptogenesis. Cell 139, 380-392 (2009).
38. Neely, G. G. et al. A genome-wide Drosophila screen for heat nociception identifies alpha2delta3 as an evolutionarily conserved pain gene. Cell 143, 628-638 (2010).
39. Suman-Chauhan, N., Webdale, L., Hill, D. R. & Woodruff, G. N. Characterisation of [3H]gabapentin binding to a novel site in rat brain: homogenate binding studies. Eur J Pharmacol 244, 293-301 (1993).
40. Wang, Y. & Welty, D. F. The simultaneous estimation of the influx and efflux blood-brain barrier permeabilities of gabapentin using a microdialysis-pharmacokinetic approach. Pharm Res 13, 398-403 (1996).
41. Su, T. Z., Feng, M. R. & Weber, M. L. Mediation of highly concentrative uptake of pregabalin by L-type amino acid transport in Chinese hamster ovary and Caco-2 cells. The Journal of pharmacology and experimental therapeutics 313, 1406-1415 (2005).
42. Radulovic, L. L. et al. Disposition of gabapentin (neurontin) in mice, rats, dogs, and monkeys. Drug metabolism and disposition: the biological fate of chemicals 23, 441-448 (1995).
43. Akat, H. et al. Biological distribution of iodo-allyl gabapentin and iodo-gabapentin. Journal of Radioanalytical and Nuclear Chemistry 275, 387-393 (2008).
44. Guo, X., Zhu, H. & Yang, Z. Novel 64Cu-NOTA-1B50-1 targeting α2δ-1 for specific cancer stem cell imaging: A preliminary experiments on Hepatocellular Carcinoma. Journal of Nuclear Medicine 60, 610-610 (2019).
45. Taylor, C. P. & Garrido, R. Immunostaining of rat brain, spinal cord, sensory neurons and skeletal muscle for calcium channel alpha2-delta (alpha2-delta) type 1 protein. Neuroscience 155, 510-521 (2008).
46. Field, M. J., Holloman, E. F., McCleary, S., Hughes, J. & Singh, L. Evaluation of gabapentin and S-(+)-3-isobutylgaba in a rat model of postoperative pain. The Journal of pharmacology and experimental therapeutics 282, 1242-1246 (1997).
47. Detloff, M. R., Fisher, L. C., Deibert, R. J. & Basso, D. M. Acute and chronic tactile sensory testing after spinal cord injury in rats. J Vis Exp, e3247 (2012).
48. Challa, S. R. Surgical animal models of neuropathic pain: Pros and Cons. Int J Neurosci 125, 170-174 (2015).
49. Akaike, H. A new look at the statistical model identification. IEEE Transactions on Automatic Control 19, 716-723 (1974).
50. Logan, J. Graphical analysis of PET data applied to reversible and irreversible tracers. Nucl Med Biol 27, 661-670 (2000).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A compound of Formula (I) or Formula (II): 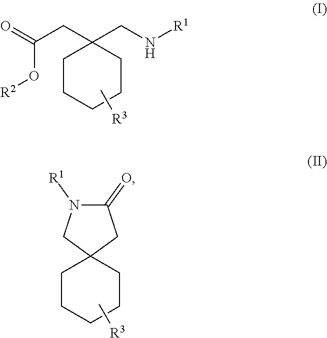
or a pharmaceutically acceptable salt thereof, wherein:
each R¹ is independently selected from H and C(═O)(O—C₁₋₆ alkyl), wherein said C₁₋₆ alkyl is optionally substituted with OC(═O)C₁₋₆ alkyl;
R² is selected from H and C₁₋₆ alkyl, benzyl; and
each R³ is independently a fluorine atom selected from F and ¹⁸F.

2. The compound of claim 1, wherein at least one hydrogen atom is a deuterium isotope.

3. The compound of claim 1, wherein at least one carbon atom is an ¹¹C isotope.

4. The compound of claim 1, wherein the fluorine atom is ¹⁸F.

5. The compound of claim 1, wherein the compound of Formula (I) is: 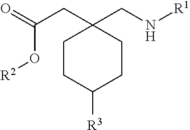
or a pharmaceutically acceptable salt thereof.

6. The compound of claim 1, wherein the compound of Formula (II) is: 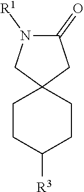
or a pharmaceutically acceptable salt thereof.

7. The compound of claim 1, wherein the compound is cis-[¹⁸F]4-fluoro-gabapentin.

8. The compound of claim 1, wherein the compound is trans-[¹⁸F]4-fluoro-gabapentin.

9.-11. (canceled)

12. A composition comprising a mixture of cis-[¹⁸F]4-fluoro-gabapentin and trans-[¹⁸F]4-fluoro-gabapentin.

13. A method of treating neuropathic pain, epilepsy, anxiety, or ataxia in a subject, the method comprising administering a therapeutically effective amount of the composition of claim 12 to a subject in need thereof.

14.-15. (canceled)

16. The method of claim 13, wherein the composition is administered at doses ranging from 0.001 mg/kg to 100 mg/kg.

17. The method of claim 13, wherein the composition is administered orally or intravenously.

18.-47. (canceled)

48. The compound of claim 1, wherein the compound of Formula (I) is: 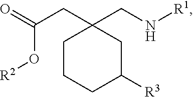
or a pharmaceutically acceptable salt thereof.

49. The compound of claim 1, wherein the compound of Formula (II) is: 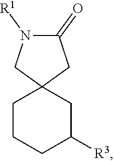
or a pharmaceutically acceptable salt thereof.

50. The compound of claim 1, wherein the compound is a mixture of cis-[¹⁸F]4-fluoro-gabapentin and trans-[¹⁸F]4-fluoro-gabapentin.

51. The compound of claim 1, wherein the one R³ in the compound of Formula (I) or Formula (II) is ¹⁸F.

52. The compound of claim 1, wherein one hydrogen atom is a deuterium isotope.

53. The compound of claim 1, wherein one carbon atom is an ¹¹C isotope.
